(19) United States
(12) Patent Application Publication
(10) Pub. No.: US 2017/0313885 A1
(43) Pub. Date: Nov. 2, 2017

(54) ALKOXYSILYL GROUP-CONTAINING ORGANIC EL DYE AND A METHOD FOR PRODUCING THE SAME

(71) Applicant: Shinichiro, Fukuoka (JP)

(72) Inventors: Shinichiro ISOBE, Fukuoka-shi, Fukuoka (JP); Shuntaro MATAKA, Onojo-shi, Fukuoka (JP)

(73) Assignee: Shinichiro, Fukuoka (JP)

(21) Appl. No.: 15/522,121

(22) PCT Filed: Oct. 30, 2015
(86) PCT No.: PCT/JP2015/080820

§ 371 Date: Apr. 26, 2017

(30) Foreign Application Priority Data
Oct. 31, 2014 (JP) 2014-223403

(51) Int. Cl. C09B 69/00; C09K 11/06; G01N 33/52

(52) U.S. Cl. CPC C09B 69/008; G01N 33/52; C09K 11/06; C09K 2211/1022

(57) ABSTRACT

Provided is an alkoxysilyl group-containing organic EL dye that can be used for the production of fluorescent silica particles that do not fade. This alkoxysilyl group-containing organic EL dye is represented by the general formula X—Y-Q-Z—Si(R₁)ₙ(OR₂)₃₋ₙ, wherein X is an organic EL dye, Y is a direct bond or —(CH₂)ₚ— (p represents an integer from 1 to 10) or —(O—CH₂CH₂)_{q}— (q represents an integer from 1 to 10), Q is a bond selected from the group consisting of amide bond, ether bond, thioether bond, thioester bond, thiourea bond, disulfide bond, and polyoxyethylene bond, Z is —(CH₂)₃— or —(CH₂)₂NH(CH₂)₃—, R₁ and R₂ represent an alkyl group having 1 to 7 carbon atoms, and n represents 0 or 1.

### TECHNICAL FIELD

The present invention relates to an alkoxysilyl group containing organic EL dye and a method for producing the same, and more particularly to an alkoxysilyl group-containing organic EL dye used for producing an alkoxysilyl group-containing silica particles used for detection of a biomolecule such as nucleic acids, proteins, peptides, and saccharides, and a method for producing the same.

### BACKGROUND ART

In recent years, the whole contents of the human genome have been clarified and post genome studies have been enthusiastically made for the purposes of executing gene therapy, genetic diagnosis and the like. For example, in DNA analysis, a DNA probe fixed onto a DNA microarray substrate is hybridized with a sample DNA labeled by a fluorescent dye or the like to form a double stranded DNA, thereby detecting the sample DNA. This is a technique in which measurement is made after a nucleic acid labeled by a fluorescent dye is PCR-extended and hybridized on a substrate. In these days, a technique using a primer having more amino groups and a technique in which an amino group is introduced into DNA are used.

A fluorescent dye is widely used for labeling, but there is a problem that the fluorescent dye easily quenches in aqueous solution. Against this problem, there has been proposed a method of suppressing the quenching of the fluorescent dye and increasing fluorescent intensity by using a fluorescent dye-containing silica particles (hereinafter referred to as fluorescent silica particles) in which the fluorescent dye is introduced (for example, Patent Document 1). Patent Document 1 discloses a method for producing fluorescent monodispersed nanoparticles comprising: providing tetramethylrhodamine isothiocyanate (TRITC) as an organic dye molecule; mixing TRITC with an organosilane compound to form a dye precursor, mixing the dye precursor with an aqueous solution to form a dense fluorescent core; and mixing the dense fluorescent core with the silica precursor to form a silica shell on the dense core.

### PATENT DOCUMENT

Patent Document 1: Japanese Laid-Open Patent Publication No. 2006-514708

### DISCLOSURE OF THE INVENTION

### Problem to be Solved by the Invention

However, there is a problem that conventional fluorescent silica particles fade easily in a dry state and a sufficient fluorescent intensity cannot be obtained.

Accordingly, an object of the present invention is to provide an alkoxysilyl group containing organic EL dye and a method for producing the same used for producing fluorescent silica particles which hardly fade in a dry state.

### Solutions to the Problem

In order to solve the above problem, an alkoxysilyl group containing organic EL dye of the present invention is characterized by being represented by the general formula X—Y-Q-Z—Si(R₁)ₙ(OR₂)₃₋ₙ, wherein X is an organic EL dye, Y is a direct bond or —(CH₂)ₚ— (p represents an integer from 1 to 10) or —(O—CH₂CH₂)_{q}— (q represents an integer from 1 to 10), Q is a bond selected from the group consisting of amide bond, ether bond, thioether bond, thioester bond, thiourea bond, disulfide bond, and polyoxyethylene bond, Z is —(CH₂)₃— or —(CH₂)₂NH(CH₂)₃—, R₁ and R₂ represent an alkyl group having 1 to 7 carbon atoms, and n represents 0 or 1.

Further, a production method of the present invention is a method for producing the above alkoxysilyl group containing organic EL dye, the method is characterized by including a step of mixing the organic EL dye with a silane coupling agent, wherein the organic EL dye includes a reactive group selected from the group consisting of a succinimidyl ester group, an alcoholate group, an amino group, a mercapto group, hydroxyl-terminated polyoxyethylene group.

Further, the fluorescent silica particles of the present invention are characterized by including condensates of the above alkoxysilyl-containing organic EL dye.

### Effects of the Invention

The alkoxysilyl group containing organic EL dye of the present invention enables to produce fluorescent silica particles having high fluorescent intensity in a dry state. Further, since the excitation wavelength and the emission wavelength of the organic EL dye can be changed by changing the substituent of the organic EL dye, the degree of freedom in the selection of fluorescent wavelength and many fluorescent wavelength such as red, orange, yellow, green, and blue can be used. This makes it possible to use two or more of fluorescent dyes having a large Stokes shift (a large difference between the excitation wavelength and the fluorescent wavelength), and also to simultaneously detect a plurality of target nucleic acids contained in one sample.

### BRIEF DESCRIPTION OF THE DRAWINGS

FIG. 1 is a fluorescence micrograph which shows the result of a fading test of the alkoxysilyl group containing organic EL dye of the present invention.

### BEST MODE FOR CARRYING OUT THE INVENTION

Embodiments of the present invention will be described in detail below.

The alkoxysilyl group containing organic EL dye of the present invention is characterized by being represented by the general formula X—Y-Q-Z—Si(R₁)ₙ(OR₂)₃₋ₙ, wherein X is an organic EL dye, Y is a direct bond or —(CH₂)ₚ— (p represents an integer from 1 to 10) or —(O—CH₂CH₂)_{q}— (q represents an integer from 1 to 10), Q is a bond selected from the group consisting of amide bond, ether bond, thioether bond, thioester bond, thiourea bond, disulfide bond, and polyoxyethylene bond, Z is —(CH₂)₃— or —(CH₂)₂NH(CH₂)₃—, R₁ and R₂ represent an alkyl group having 1 to 7 carbon atoms, and represents 0 or 1.

The organic EL dye used in the present invention is not particularly limited provided it is a dye sandwiched in solid state between a pair of anode and cathode and capable of emitting by virtue of energy in recombination of a hole injected from an anode and an electron injected from a cathode. For example, poly-ring aromatic compounds such as tetraphenylbutadiene, perylene and the like, cyclopentadiene derivatives, distyrylpyrazine derivatives, acridone derivatives, quinacridone derivatives, stilbene derivatives, phenothiazine derivatives, pyradinopyridine derivatives, diazolopyridine derivatives, imidazole derivatives, carbazole derivatives, tetraphenylthiophene derivatives and the like can be used. Preferable compounds are diazolopyridine derivatives, imidazole derivatives or carbazole derivatives.

By changing the substituent of the organic EL dye used in the present invention, it is possible to change the excitation wavelength and the emission wavelength, and thereby obtaining emission colors of red, blue and green.

The above Y represents a direct bond or —(CH₂)ₚ— (p represents an integer of 1 to 10) or —(O—CH₂CH₂)_{q}— (q represents an integer of 1 to 10). p represents preferably 1 to 8, or more preferably 1 to 4. Further, q represents preferably 1 to 8, or more preferably 1 to 4.

Further, Q is a bond selected from the group consisting of amide bond, ether bond, thioether bond, thioester bond, thiourea bond, disulfide bond, and polyoxyethylene bond. Q is preferably amid bond or polyoxyethylene bond. The amide bond can be represented by —CO(NR)—, wherein R represents hydrogen or an alkyl group having 1 to 4 carbon atoms, preferably R represents hydrogen or methyl group. Further, polyoxyethylene bond can be represented by —(O—CH₂CH₂)ᵣ—, r represents an integer of 1 to 10, preferably an integer of 1 to 5.

Further, the above Z represents —(CH₂)₃— or —(CH₂)₂NH(CH₂)₃—, preferably —(CH₂)₃—.

Further, R₁ and R₂ of Si(R₁)ₙ(OR₂)₃₋ₙ represent alkyl group having 1 to 4 carbon atoms, preferably methyl group, ethyl group, n-propyl group, more preferably methyl group or ethyl group. n represents 0 or 1.

Further, the alkoxysilyl group-containing organic EL dye can be produced by the following method. That is, the method is characterized by including a step of mixing the organic EL dye with a silane coupling agent, wherein the organic EL dye includes a reactive group selected from the group consisting of a succinimidyl ester group, an alcoholate group, an amino group, a mercapto group, hydroxyl-terminated polyoxyethylene group.

The organic EL dye used in the present invention includes the above reactive group and reacts with the silane coupling agent to form a covalent bond, and thereby bonding with the silane coupling agent. The covalent bond is amide bond, ether bond, thiourea bond, disulfide bond or polyoxyethylene bond. When forming the amide bond, aminoalkylsilane can be used as the silane coupling agent and a succinimidyl ester group can be used as the reactive group of the organic EL dye. When forming the ether bond, a halogenated alkylsilane can be used as the silane coupling agent and an alcoholate group can be used as the reactive group of the organic EL dye. Further, when forming the thiourea bond, isothiocyanate can be used as the silane coupling agent and an amide group can be used as the reactive group of the organic EL dye. Further, when forming the disulfide bond, mercaptosilane can be used as the silane coupling agent and a mercapto group can be used as the reactive group of the organic EL dye. Further, when forming the polyoxyethylene bond, glycidyloxyalkylsilane is used as the silane coupling agent and hydroxyl terminated polyoxyethylene group as the reactive group of the organic EL dye.

As the silane coupling agent, aminoalkylsilane, glycidyloxyalkylsilane, mercaptosilane, isothiocynate, isocyanate silane, halogenated silane and the like can be used. Preferable coupling agent is aminoalkylsilane. Specific examples of the aminoalkylsilane includes 3-aminopropyltriethoxysilane, 3-(2-aminoethylamino)propyltrimethoxysilane, 3-(2-aminoethylamino)propyldimethoxymethlysilane, 3-(2-aminoethylamino)propyltriethoxysilane), 3-aminopropyldiethoxymethylsilane, 3-aminopropyltrimethoxysilane and the like, preferably 3-aminopropyltrimethoxysilane. Further, specific examples of the glycidyloxyalkylsilane include diethoxy(3-glycidyloxypropyl)methylsilane, 3-glycidyloxypropyltrimethoxysilane, 3-glycidyloxypropyl(dimethoxy)methylsilane, trietoxy(3-glycidyloxypropy)silane and the like. Further, specific examples of the mercaptosilane include 3-mercaptopropylmethylmethoxysilane and 3-mercaptopropyltrimethoxysilane. Further, specific examples of the isocyanate silane include 3-isocyanatepropyltriethoxysilane and 3-isocyanatepropyltrimethoxysilane. Further, specific examples of the isothiocyanate include 3-thiocyanatepropyltriethoxysilane. Further, specific examples of the halogenated silane include (3-bromopropyl)trimethoxysilane, 3-trimethoxysilylpropyl chloride, 3-chloropropyldimethoxymethylsilane, 3-iodopropyltrimethoxysilane and the like.

The reaction between the organic EL dye and the silane coupling agent can be carried out by mixing and stirring the mixture at room temperature to 60° C. in solvent such as dichloromethane, chloroform or DMF. The reactant can be taken out by removing the solvent using such as decompression as needed.

Further, fluorescent silica particles can be produced by using the alkoxysilyl group-containing organic EL dye of the present invention. The method for producing the fluorescent silica particles is not particularly limited as long as it is a method for producing silica particles using a silane coupling agent. For example, a method described in the Patent Document 1 can be used, in which a dense fluorescent core is formed by mixing an alkoxysilyl group-containing organic EL dye with an aqueous solution, and the dense fluorescent core and the silica precursor are mixed to form a silica shell on the dense fluorescent core.

### Embodiment 1

Diazolopyridine derivatives represented by the following general formulae (1), (2) and (3) are used as the organic EL dye of the alkoxysilyl group-containing organic EL dye of this embodiment.

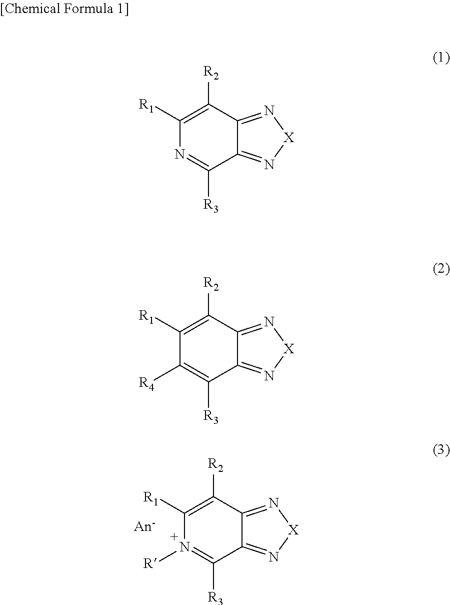

R₁ in the formulae (1) and (3) and one of R₁ and R₄ in the formula (2) are each represented by the general formula L₁-M₁, wherein M₁ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent, L₁ represents a linker which is represented by —(CH═CR₆)ₛ— and which connects M₁ with a center pyridine ring or a center benzene ring, s represents an integer of from 1 to 5, R₆ represents any one of a hydrogen atom; a linear or branched alkyl group which may have a substituent and has 1 to 6 carbon atoms; a sulfo group which may have a substituent; a heterocyclic group selected from the group consisting of an imidazolium group, a pyridinium group, and a furan group, each of which may have a substituent; an amino group selected from the group consisting of a secondary amino group, a tertiary amino group, and a quaternary amino group, each of which may have a substituent; a hydroxy group which may have a substituent; an alkoxy group which may have a substituent; an aldehyde group which may have a substituent; a carboxyl group which may have a substituent; and an aromatic group which may have a substituent, the residues of R₁ and R₄ in the formula (2) and R₂ and R₃ in the formulae (1) to (3) each independently represent a hydrogen atom, a halogen atom, or an aromatic hydrocarbon group, an aliphatic hydrocarbon group, or a heterocyclic group, each of which may have a substituent, X represents a nitrogen atom, a sulfur atom, an oxygen atom, a selenium atom, or a boron atom, each of which may have a substituent, R′ represents an aliphatic hydrocarbon group including an alkyl group which may have an aromatic ring or an aromatic hydrocarbon group, and An⁻ represents a halide ion, CF₃SO₃⁻, BF₄⁻, or PF₆⁻.

M₁ is directly bonded to the above linking group Q, or indirectly bonded to the above linking group through —(CH₂)ₚ— (p represents an integer of 1 to 10) or —(O—CH₂CH₂)_{q}— (q represents an integer of 1 to 10).

R₂ and R₃ represent each independently preferably an aromatic hydrocarbon group, an aliphatic hydrocarbon group, or a heterocyclic group, each of which may have a substituent. Examples of the substituent may include: an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms. The aromatic hydrocarbon group as the substituent is an aryl group containing a monocycle or polycycle. The heterocyclic group as the substituent is, for example, a thienyl group, a furanyl group, a pyrrolyl group, an imidazolyl group, an oxazolyl group, a thiadiazolyl group, a pyrazolyl group, a pyridyl group, or a quinolyl group. R₂ and R₃ may each be an aryl group having a sulfonyl group.

R₂ and R₃ above represent each independently preferably a thienyl group, a furanyl group, a pyrrolyl group, an imidazolyl group, an oxazolyl group, a thiadiazolyl group, a pyrazolyl group, a pyridyl group, or a quinolyl group, each of which may have a substituent. This is because the fluorescent wavelength is shifted to the large wavelength side to obtain a larger stokes shift as compared with the case of using an unsubstituted group or a phenyl group. R₂ and R₃ each more preferably represent a thienyl group which may have a substituent, wherein the substituent is an aromatic hydrocarbon group, an aliphatic hydrocarbon group, or a heterocyclic group, each of which may have a substituent. Here, the aromatic hydrocarbon group which may have a substituent is an aryl group containing a monocycle or a polycycle, and examples of the aryl group may include a substituted or unsubstituted phenyl group, a naphthyl group, a biphenyl group and the like. In this case, the aromatic hydrocarbon group which may have a substituent may include the substituents in the number of 1 to 3. Examples of the substituents may include an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms. Examples of the aliphatic hydrocarbon group which may have a substituent may include a substituted or unsubstituted, linear or branched alkyl group having 1 to 20 carbon atoms, an unsubstituted, linear or branched alkenyl group having 2 to 20 carbon atoms, and an unsubstituted, linear or branched alkynyl group having 2 to 20 carbon atoms and the like. Examples of the heterocyclic group which may have a substituent may include substituted or unsubstituted furanyl group, pyrrolyl group, imidazolyl group, oxazolyl group, thiadiazolyl group, pyrazolyl group, pyridyl group, and quinolyl group. As the substituent of the thienyl group, an aromatic hydrocarbon group or an aliphatic hydrocarbon group which may have a substituent is preferable, an aromatic hydrocarbon group which may have a substituent is more preferable, and an aryl group having a monocycle or polycycle is furthermore preferable. Specific examples may include substituted or unsubstituted phenyl group, naphthyl group, biphenyl group and the like. In this case, the aromatic hydrocarbon group which may have a substituent may include the substituents in the number of 1 to 3, and examples of the substituent may include an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms.

Further, the above reactive group may be the above M₁ or may be introduced into M₁.

According to this embodiment, the alkoxysilyl group-containing EL dye, which is obtained by reacting the above diazolopyridine derivative with the above silane coupling agent, hardly fades in a dry state. Therefore, it is possible to provide fluorescent silica particles which hardly fade in a dry state by producing the fluorescent silica particles using the alkoxysilyl group-containing EL dye. Further, since the diazolopyridine derivative used in this embodiment has high water solubility, and therefore the rate of labeling for a biomolecule can be improved to thereby detect a biomolecule with high sensitivity. The excitation wavelength and emission wavelength are altered by changing the substituent of the diazolopyridine derivative to enable the use of two or more types of the fluorescent silica particles each having a large stokes shift, thereby making it possible to simultaneously detect a plurality of target molecules contained in one sample. When, particularly, a thienyl group which may have a substituent is used as R₂ and R₃, a stokes shift exceeding 100 nm is obtained, and therefore detection with high sensitivity can be performed without any influence of excitation light.

### Embodiment 2

Diazolopyridine derivatives represented by the following general formulae (4), (5) and (6) are used as the organic EL dye of the alkoxysilyl group-containing organic EL dye of this embodiment. An effect similar to that in the case of the Embodiment 1 can be obtained in this embodiment.

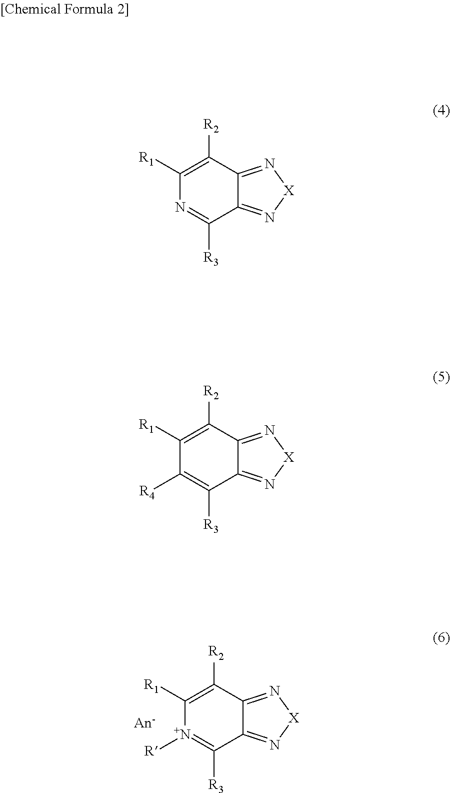

R₁ in the formulae (4) and (6) and one of R₁ and R₄ in the formula (5) are each represented by the general formula L₂-M₂, wherein M₂ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent, L₂ represents a linker which connects M₂ with a center pyridine ring or a center benzene ring and which represents a direct bond or one or more types of functional groups selected from the group consisting of —(CH₂)ₜ— (t represents an integer of from 1 to 4), —NHCOO—, —CONH—, —CON(CH₃)—, —COO—, —SO₂NH—, —HN—C(═NH)—NH—, —O—, —S—, —NR (R represents an alkyl group), —Ar— (Ar represents an aromatic hydrocarbon group), and —CO—Ar—NR—, the residues of R₁ and R₄ in the formula (5) and R₂ and R₃ in the formulae (4) to (6) each independently represent a thienyl group, a furanyl group, a pyrrolyl group, an imidazolyl group, an oxazolyl group, a thiadiazolyl group, a pyrazolyl group, a pyridyl group, or a quinolyl group, each of which may have a substituent, X represents a nitrogen atom, a sulfur atom, an oxygen atom, a selenium atom, or a boron atom, each of which may have a substituent, R′ represents an aliphatic hydrocarbon group including an alkyl group which may have an aromatic ring or an aromatic hydrocarbon group, and An⁻ represents a halide ion, CF₃SO₃⁻, BF₄⁻, or PF₆⁻.

M₂ is directly bonded to the above linking group Q, or indirectly bonded to the above linking group through —(CH₂)ₚ— (p represents an integer of 1 to 10) or —(O—CH₂CH₂)_{q}— (q represents an integer of 1 to 10).

R₂ and R₃ each more preferably represent a thienyl group which may have a substituent, wherein the substituent is an aromatic hydrocarbon group, an aliphatic hydrocarbon group, or a heterocyclic group, each of which may have a substituent. Here, the aromatic hydrocarbon group which may have a substituent is an aryl group containing a monocycle or a polycycle, and specific examples of the aryl group may include a substituted or unsubstituted phenyl group, a naphthyl group, a biphenyl group and the like. In this case, the aromatic hydrocarbon group which may have a substituent may include the substituents in the number of 1 to 3. Examples of the substituents may include an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms. Examples of the aliphatic hydrocarbon group which may have a substituent may include a substituted or unsubstituted, linear or branched alkyl group having 1 to 20 carbon atoms, an unsubstituted, linear or branched alkenyl group having 2 to 20 carbon atoms, and an unsubstituted, linear or branched alkynyl group having 2 to 20 carbon atoms and the like. Examples of the heterocyclic group which may have a substituent may include substituted or unsubstituted furanyl group, pyrrolyl group, imidazolyl group, oxazolyl group, thiadiazolyl group, pyrazolyl group, pyridyl group, and quinolyl group. As the substituent of the thienyl group, an aromatic hydrocarbon group or an aliphatic hydrocarbon group which may have a substituent is preferable, and an aromatic hydrocarbon group which may have a substituent is more preferable, and specific examples may include substituted or unsubstituted phenyl group, naphthyl group, biphenyl group and the like. In this case, the aromatic hydrocarbon group which may have a substituent may include the substituents in the number of 1 to 3, and examples of the substituent may include an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms.

Further, the above reactive group may be the above M₂ or may be introduced into M₂.

### Embodiment 3

Diazolopyridine derivatives represented by the following general formulae (7), (8) and (9) are used as the organic EL dye of the alkoxysilyl group-containing organic EL dye of this embodiment. An effect similar to that in the case of the Embodiment 1 can be obtained in this embodiment.

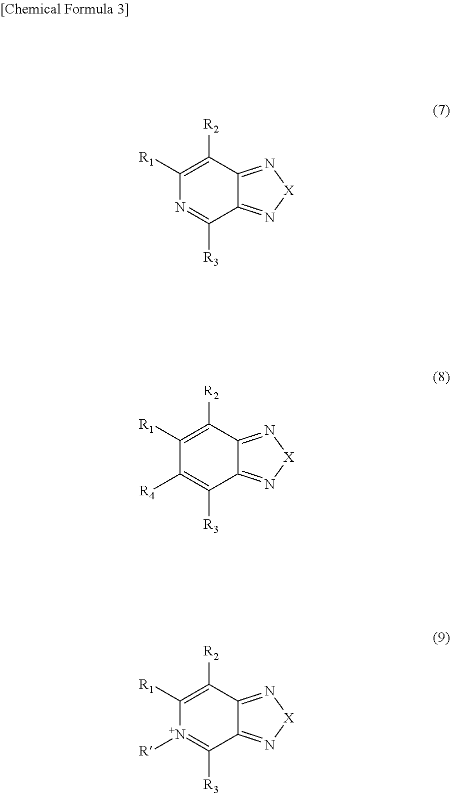

R₁, R₂, R₃ and R₄ in the formula (7), (8) and (9) each independently represent a hydrogen atom, a halogen atom, or an aromatic hydrocarbon group, or an aliphatic hydrocarbon group, or a heterocyclic group, each of the aromatic hydrocarbon group, the aliphatic hydrocarbon group and the heterocyclic group may have a substituent selected from the group consisting of an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group, X represents a nitrogen atom, a sulfur atom, an oxygen atom, a selenium atom, or a boron atom, each of which may have a substituent, R′ represents an aliphatic hydrocarbon group including an alkyl group which may have an aromatic ring or an aromatic hydrocarbon group, and An⁻ represents a halide ion, CF₃SO₃⁻, BF₄⁻, or PF₆⁻.

R₁ or R₄ is directly bonded to the above linking group.

R₂ and R₃ above are each independently preferably a thienyl group, a furanyl group, a pyrrolyl group, an imidazolyl group, an oxazolyl group, a thiadiazolyl group, a pyrazolyl group, a pyridyl group, or a quinolyl group, each of which may have a substituent.

R₂ and R₃ each more preferably represent a thienyl group which may have a substituent, wherein the substituent is an aromatic hydrocarbon group, an aliphatic hydrocarbon group, or a heterocyclic group, each of which may have a substituent.

The above reactive group may be introduced into R₁ or R₄.

### Embodiment 4

Imidazole derivatives represented by the following general formulae (10)-(14) are used as the organic EL dye of the alkoxysilyl group-containing organic EL dye of this embodiment. An effect similar to that in the case of the Embodiment 1 can be obtained in this embodiment.

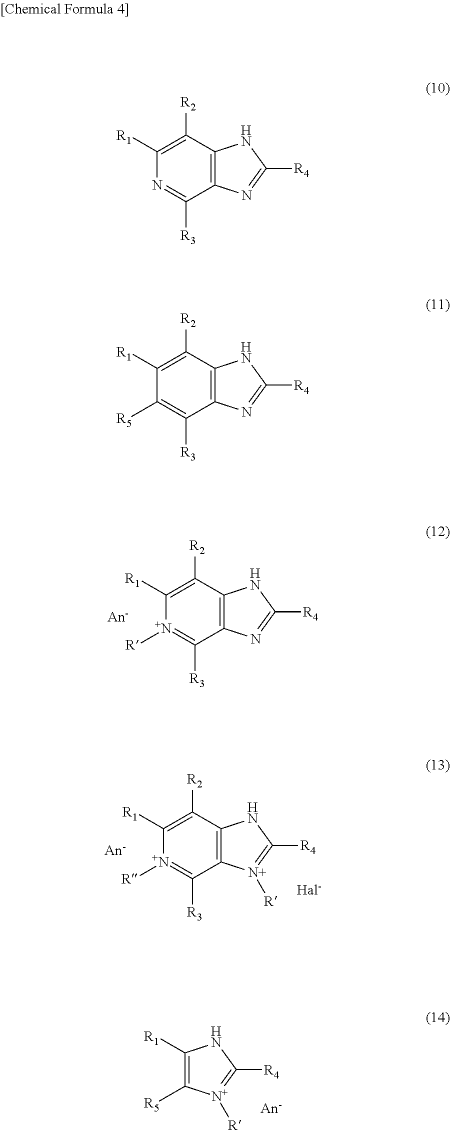

One of R₁ and R₄ in the formulae (10), (12) and (13) and one of R₁, R₄ and R₅ in the formulae (11) and (14) are each represented by the general formula L₃-M₃, wherein M₃ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent, L₃ represents a linker which is represented by —(CH═CR₆)ᵤ— and which connects M₃ with a center pyridine ring or a center benzene ring, u represents an integer of from 1 to 5, R₆ represents any one of a hydrogen atom; a linear or branched alkyl group which may have a substituent and has 1 to 6 carbon atoms; a sulfo group which may have a substituent; a heterocyclic group selected from the group consisting of an imidazolium group, a pyridinium group, and a furan group, each of which may have a substituent; an amino group selected from the group consisting of a secondary amino group, a tertiary amino group, and a quaternary amino group, each of which may have a substituent; a hydroxy group which may have a substituent; an alkoxy group which may have a substituent; an aldehyde group which may have a substituent; a carboxyl group which may have a substituent; and an aromatic group which may have a substituent, the residues of R₁ and R₄ in the formula (10), (12) and (13), the residues of R₁, R₄ and R₅ in the formulae (11) and (14), and R₂ and R₃ each independently represent a hydrogen atom, a halogen atom, or an aromatic hydrocarbon group, an aliphatic hydrocarbon group, or a heterocyclic group, each of which may have a substituent, X represents a nitrogen atom, a sulfur atom, an oxygen atom, a selenium atom, or a boron atom, each of which may have a substituent, R′ represents an aliphatic hydrocarbon group including an alkyl group which may have an aromatic ring or an aromatic hydrocarbon group, and An⁻ represents a halide ion, CF₃SO₃⁻, BF₄⁻, or PF₆⁻.

M₃ is directly bonded to the above linking group Q, or indirectly bonded to the above linking group through —(CH₂)ₚ— (p represents an integer of 1 to 10) or —(O—CH₂CH₂)_{q}— (q represents an integer of 1 to 10).

R₂ and R₃ are each independently preferably an aromatic hydrocarbon group, an aliphatic hydrocarbon group, or a heterocyclic group, each of which may have a substituent. Examples of the substituent may include: an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms. The aromatic hydrocarbon group as the substituent is an aryl group containing a monocycle or polycycle. The heterocyclic group as the substituent is, for example, a thienyl group, a furanyl group, a pyrrolyl group, an imidazolyl group, an oxazolyl group, a thiadiazolyl group, a pyrazolyl group, a pyridyl group, or a quinolyl group. R₂ and R₃ may each be an aryl group having a sulfonyl group.

R′ and R″ each represent an aliphatic hydrocarbon group or an aromatic hydrocarbon group including an alkyl group which may have an aromatic ring. Here, as the aliphatic hydrocarbon group or the aromatic hydrocarbon group, ones similar to those described above may be used.

R₂ and R₃ above are each independently preferably a thienyl group, a furanyl group, a pyrrolyl group, an imidazolyl group, an oxazolyl group, a thiadiazolyl group, a pyrazolyl group, a pyridyl group, or a quinolyl group, each of which may have a substituent, similarly to the case of Embodiment 1. This is because the fluorescent wavelength is shifted to the large wavelength side to obtain a larger stokes shift as compared with the case of using an unsubstituted group or a phenyl group. R₂ and R₃ each more preferably represent a thienyl group which may have a substituent, wherein the substituent is an aromatic hydrocarbon group, an aliphatic hydrocarbon group, or a heterocyclic group, each of which may have a substituent. As the substituent of the thienyl group, substituents similar to those in the case of Embodiment 1 may be used.

Further, the above reactive group may be the above M₃ or may be introduced into M₃.

### Embodiment 5

Carbazole derivatives represented by the following general formula (15) are used as the organic EL dye of the alkoxysilyl group-containing organic EL dye of this embodiment. An effect similar to that in the case of the Embodiment 1 can be obtained in this embodiment.

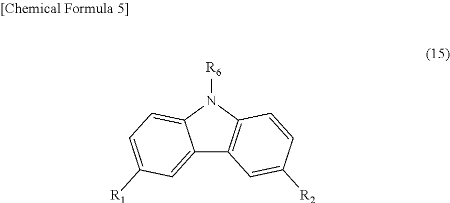

R₁ and R₂ are each independently a hydrogen atom, a halogen atom, or an aromatic hydrocarbon group, or an aliphatic hydrocarbon group, or a heterocyclic group, each of the aromatic hydrocarbon group, the aliphatic hydrocarbon group and the heterocyclic group may have a substituent. Examples of the substituent may include: an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group. The alkyl group as the substituent is a substituted or unsubstituted, linear or branched alkyl group having 1 to 20 carbon atoms. The alkenyl group as the substituent is an unsubstituted, linear or branched alkenyl group having 2 to 20 carbon atoms. The alkynyl group as the substituent is an unsubstituted, linear or branched alkynyl group having 2 to 20 carbon atoms. The alkoxy group as the substituent is, for example, a methoxy group, an ethoxy group, a propoxy group, an isopropoxy group, a butoxy group, a pentyloxy group, or a phenoxy group. The alkyl ester group as the substituent is a linear or branched alkyl ester having 1 to 6 carbon atoms. The aromatic hydrocarbon group as the substituent is an aryl group containing a monocycle or polycycle. The heterocyclic group as the substituent is, for example, a thienyl group, a furanyl group, a pyrrolyl group, an imidazolyl group, an oxazolyl group, a thiadiazolyl group, a pyrazolyl group, a pyridyl group, or a quinolyl group. R₂ and R₃ may each be an aryl group having a sulfonyl group.

R₅ represents an aliphatic hydrocarbon group or an aromatic hydrocarbon group including an alkyl group which may have an aromatic ring.

R₁ or R₂ may be directly bonded to the above linking group Q.

The above reactive group may be introduced into R₁ or R₂.

### Embodiment 6

The alkoxysilyl group-containing organic EL dye of this embodiment is a diazolopyridine derivative represented by the general formulae (1), (2) and (3), and R₁ in the formulae (1), (3) and one of R₁ and R₄ in the formula (2) are directly bonded to the above linking group Q. The linking group Q may be selected from the group consisting of amide bond, ether bond, thioether bond, thioester bond, thiourea bond, disulfide bond, and polyoxyethylene bond. Then, the linking group Q is bonded to the alkoxysilyl group through the above Z. The residue of R₁ and R₄ in the formula (2) and R₂ and R₃ in the formulae (1)-(3) are each independently a hydrogen atom, a halogen atom, or an aromatic hydrocarbon group, or an aliphatic hydrocarbon group, or a heterocyclic group, each of the aromatic hydrocarbon group, the aliphatic hydrocarbon group and the heterocyclic group may have a substituent. X in the formulae (1), (2) and (3) represents a nitrogen atom, a sulfur atom, an oxygen atom, a selenium atom, or a boron atom, each of which may have a substituent, R′ represents an aliphatic hydrocarbon group including an alkyl group which may have an aromatic ring or an aromatic hydrocarbon group, and An⁻ represents a halide ion, CF₃SO₃⁻, BF₄⁻, or PF₆⁻.

The alkoxysilyl group-containing organic EL dye of this embodiment, which is obtained by reacting the above diazolopyridine with the above silane coupling agent, hardly fade in a dry state. Therefore, it is possible to provide fluorescent silica particles which hardly fade in a dry state by producing the fluorescent silica particles using the alkoxysilyl group-containing organic EL dye of this embodiment.

The fluorescent silica particles can be produced by mixing the alkoxysilyl group-containing organic EL dye of present invention with an aqueous solution and condensing. For example, a method described in the Patent Document 1 can be used, in which a dense fluorescent core is formed by mixing the alkoxysilyl group-containing organic EL dye with the aqueous solution, and the dense fluorescent core and the silica precursor are mixed to form a silica shell on the dense fluorescent core.

### (Application)

The fluorescent silica particles of present invention can be applied to any method for detection of a biomolecule provided it is a method of measuring the fluorescence of a labeled biomolecule in solid or semi-solid state, and the following applications can be expected. For example, the fluorescent silica particles of present invention can be used in DNA micro-array method for detecting nucleic acid and PCR method using such as a primer or terminator.

Where the object to be detected is a protein, a chromatic dye is used for the detection of the protein after electrophoresis. Generally, a method comprising penetrating a chromatic dye such as Coomassie Brilliant Blue (CBB) to a gel after electrophoresis to stain a protein and irradiating the protein with UV to cause luminescence is used. Although such method using a conventional chromatic dye is convenient, it is not suitable for the detection of trace protein because the sensitivity is low as about 100 ng. Furthermore, the method also has a problem in that long time is required for dying because the chromatic dye is penetrated through the gel. The fluorescent silica particles of present invention are suitable for the detection of trace protein, because it has high sensitivity. Furthermore, the protein separated by size separation can also be identified by mass analysis.

Examples of the protein that can be detected include simple proteins such as albumin, globulin, glutelin, histone, protamine, collagen, etc., and conjugated proteins such as nucleus protein, glycoprotein, riboprotein, phosphoprotein, metal protein, etc. For example, phosphoprotein, glycoprotein and whole protein can be stained in a protein sample separated by two-dimensional electrophoresis using three organic fluorescent dyes that correspond to chromatic dyes for phosphoprotein, glycoprotein and whole protein. Furthermore, since the protein can be identified by mass analysis such as TOF-Mass, etc., it can be applied to the diagnosis or treatment of diseases that produce specific protein such as cancer, infectious diseases due to virus, etc. Collagen is a protein that constitutes binding tissues of animals, and has a unique fibrous structure, i.e., a structure having three polypeptide strands in which said peptide strands aggregate to form a triple strand. Generally, collagen is a protein having quite low immunogenicity, and is widely used in the fields of foods, cosmetics, pharmaceuticals, etc. However, where a fluorescence dye is introduced in the peptide strand of collagen, its stability is insufficient where a conventional fluorescence dye is used. Therefore, a more stable fluorescence dye is required. Accordingly, stable and high sensitivity detection can be carried out by using fluorescent silica particles of the present invention for labeling collagen.

Further, a protein can be labeled by labeling an antibody specifically binding with a protein with fluorescent silica particles of the present invention. For example, a fragment that referred to as F(ab′)₂ can be obtained by treating an IgG antibody with pepsin. The fragment is reduced using dithiothreitol, etc. to give a fragment that referred to as Fab′. The Fab′ fragment has one or two thiol group(s) (—SH). Specific reaction can be carried out by reacting the thiol group(s) with maleimide group(s). Namely, an antibody can be labeled with the fluorescent silica particles of the present invention by reacting the fluorescent silica particles in which maleimide group(s) have been introduced with thiol group(s) of a fragment. In this case, the physiological activity (antibody capturing ability) of the antibody is not deteriorated.

Meanwhile, fluorescent silica particles of the present invention can be used for labeling an aptamer. Since the aptamer comprises an oligonucleic acid and can form various characteristic stereo structures depending on the base sequence, it can bind to many biomolecules including proteins via its stereo structure. Using this characteristic, the object substance can be detected by binding an aptamer labeled with the fluorescent silica particles of the present invention to a specific protein, and detecting indirectly the object substance to be detected from the variation of fluorescence according to the change of the structure of the protein due to binding to the object substance to be detected.

Alternatively, metal ion can be detected using fluorescent silica particles of the present invention. Metal ion participates to every life phenomena that occur in a living body, such as maintenance of stability and high dimension structure of DNAs, proteins, etc. in a body, expression of functions, activation of enzymes that control all chemical reactions in a living body, etc. Therefore, importance of a metal ion sensor, which can observe behavior of metal ion in a living body in real time, is growing in the field of medical. Conventionally, a metal ion sensor in which a fluorescence dye has been introduced in a biomolecule is known. For example, a metal ion sensor that utilizes a nucleic acid having a sequence that forms a specific structure by incorporating K⁺ ion in the presence of K⁺ ion has been suggested (J. AM. CHEM. SOC. 2002, 124, 14286-14287). A fluorescence dye that initiates energy transfer is introduced in both ends of a nucleic acid. Generally, energy transfer does not occur due to distance between the dyes. However, in the presence of K⁺ ion, the nucleic acid forms a specific shape, whereby the fluorescence dyes verge in a distance that occurs energy transfer and fluorescence can be observed. In addition, a zinc ion sensor in which a fluorescence dye has been introduced in a peptide has been suggested (J. Am. Chem. Soc. 1996, 118, 3053-3054). By using a label dye comprising the fluorescent silica particles of the present invention instead of these conventional fluorescence dyes, a metal ion sensor that provides high sensitivity and easy handling property can be provided. All kinds of metal ion existing in a living body can be detected.

Moreover, intercellular signal can be observed using the fluorescent silica particles of the present invention. For the response of cells to internal signal or environmental information, various molecules from ions to enzymes are participated. It is known that in the process of signal transmission, a specific protein kinase is activated and induces phosphoration of a specific cell protein, which bears initial response for various cell responses. Binding and hydrolysis of nucleotides play an important role in these activities, and signal transmission behavior can be readily observed using a nucleotide derivative. For example, protein kinase C (PKC) plays an important role for signal transmission in a cell membrane. This Ca²⁺ dependant serine/threonine protein kinase is activated on a membrane-constituting lipid such as diacylglycerol, phosphatidyl serine, etc., which phosphorizes serine and threonine existing on an ion channel and a cell skeleton protein to vary electron charge on the membrane surface, whereby signal transmission is achieved. By dynamically observing these phenomena in living cells, signal transmission of the cells can be observed.

In this observation, the nucleotide derivative is provided as a substrate or an inhibitor for an enzyme, and it is used for search for the structure and dynamics of a lone protein and reconstruction of a membrane binding protein enzyme, and binds to organelle such as mitochondria, nucleotide-binding protein portion of tissues such as skinned muscle fiber so as to control them. Furthermore, existence of compounds that affect signal transmission such as inhibitors or active forms for G-protein has been recently revealed. By introducing the fluorescent silica particles of the present invention into this nucleotide derivative, dynamic observation of the intercellular signal transmission thereof can be carried out at high sensitivity and with easy handling.

Further, the fluorescent silica particles of the present invention can also be used as a chromatic dye for tissues or cells used for determination of the expression level of the target nucleic acid or target protein in a tissue sample or a cell sample. The tissues or cells can be stained by binding the fluorescent silica particles of the present invention with a target nucleic acid or a target protein via reactive groups as mentioned above. Accordingly, the fluorescent silica particles of the present invention shows superior performance than conventional dyes in view of storage after labeling. Furthermore, it can also be sufficiently used as a dye for cell skeletons as well as a dye for eucaryotic cells. Moreover, it can be used for labeling of mitochondria, Golgi body, endoplasmic reticulumlysosome, lipid double membrane, etc. These labeled cells, etc. can be observed under all wet or dry conditions, and thus have great versatility. A fluorescence microscope, etc. can be used for observation.

Tissues collected from a human body in a clinical stage are each sliced into a thin film by using an instrument such as a microtome, and is then dyed. Here, a Cy dye and Alexa dye are used. However, the existing dyes are very unstable, and it is therefore necessary to produce a sample again in rediagnosis. The sample produced cannot be preserved as a specimen. However, the fluorescent silica particles of the present invention is very stable as compared with the above existing dyes, and therefore tissues dyed can be preserved as a specimen.

Also, an immunoassay utilizing the specific recognition performance of an antibody is used for the diagnosis of cancers, infectious diseases and the like. This immunoassay is a method of detecting an objective antigen by using a labeled antibody, and, for example, an enzyme immunoassay (ELISA method) using an enzyme as a labeled substance and a fluorescent immunoassay (FIA method) using a fluorescent dye as a labeled substance are used. In the ELISA method, final detection is attained by detecting and quantitatively measuring various signals (for example, color development, luminescence, and chemiluminescence) generated by the reaction of an enzyme serving as a labeled substance. The FIA method is, on the other hand, performed by irradiating a fluorescent dye serving as a labeled substance with excitation light to thereby detect and quantitatively measure the fluorescent light resulted from the irradiation. Since the FIA method uses a fluorescent dye, the method has the characteristics that it provides a clear contrast and excellent quantitativity, and also enables detection in a shorter time, and is performed by simple operation as compared with the ELISA method. However, a conventional fluorescent dye has a problem concerning a low labeling rate. For example, though a fluorescent dye is used in a molar amount of about 200 times based on an antibody, the rate of labeling is about 50 to 60% even in this condition. For this reason, it is necessary to use a fluorescent dye in a large amount, and therefore there is a problem concerning high detection cost and also requiring a long time for detection because of the necessity of a process for removing an unreacted fluorescent dye. Contrary to this, the use of the fluorescent silica particles of the present invention can increase the rate of labeling, enabling highly sensitive detection.

The fluorescent silica particles of the present invention may also be used for cosmetic compositions. The cosmetic compositions containing a fluorescent dye are used not only for performance cosmetics at night and in a room, but also for foundations, hair dyes, and the like by utilizing the color-brightening effect of the fluorescent dye. Here, the color-brightening effect refers to the effect that a fluorescent dye absorbs ultraviolet light and emits visible light to impart brightness and vividness to the skin and hair. Although fluorescent lamps emitting daylight color or white light are used for indoor lighting in Japan, these fluorescent lamps mainly emit blue or green light but few red light. For this reason, there is the problem that the makeup skin of female looks pale and dark. Contrary to this, a vividly reddish color can be developed to solve the dark-color problem by using the fluorescent silica particles of the present invention which emits, for example, orange light. When the fluorescent dye of the present invention is used for hair dyeing, it can not only change the color of hair by rays emitted in a visible region, but also increase the brightness of hair.

The fluorescent silica particles of the present invention may also be used for marking agents. Although the marking agent containing the fluorescent silica particles of the present invention is invisible under normal visible light, it allows the fluorescent dye to emit light by irradiating it with excitation light such as ultraviolet rays, thereby enabling visual recognition. This marking agent may be used for the discrimination of, for example, products and human bodies and for the detection of substances for the purpose of crime prevention or criminal investigation by utilizing this nature. The objects of the marking agent include products and human bodies which are the objects of prevention of crimes such as forgeries and robberies or criminal investigation. Examples of these products and human bodies may include paper money, checks, stocks, important documents such as various certificates, products such as an automobile, a motorcycle, a bicycle, a work of art, furniture, brand items, and clothes, body surface parts such as human skin, hair, and nail, materials left behind such as latent fingerprints, and the like. Further, examples of the material constituting the objects may include paper such as wood free paper, OCR paper, non-carbon paper, and art paper, plastics such as vinyl chloride, polyester, polyethylene terephthalate, and polypropylene, metals, glasses, ceramics, natural fibers such as wool, cotton, silk, and hemp, synthetic fibers such as a regenerated cellulose fiber, a polyvinyl alcohol fiber, a polyamide fiber, and a polyester fiber, proteins in the human skin and body fluid, and the like.

### EXAMPLES

The present invention will be described in more detail by way of working examples, but the scope of the present invention is not limited by the following working examples.

### Synthetic Example 1

An example of synthesis of an ester of 4,7-diphenyl-1,2,5-oxadiazolopyridine containing 3-aminopropyltrimethoxysilane (hereinafter abbreviated as APS) as an alkoxysilyl group will be described. First, the following Scheme 1 is a synthetic scheme of the ester (4).

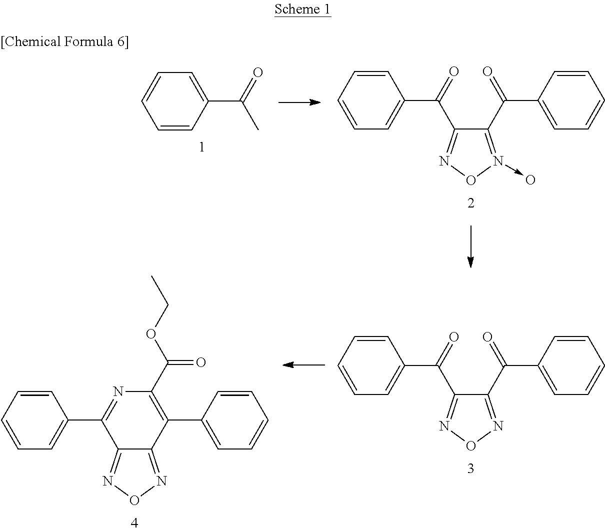

### (1) Synthesis of a Diketone Derivative (2)

In a 500 ml three-neck flask, 30.0 g (0.25 mol) of 4-methoxyacetophenone (1) and 0.15 g of sodium nitrite were dissolved in 100 ml of acetic acid. A solution prepared by dissolving 100 ml of nitric acid in 100 ml of acetic acid was added dropwise to the solution in a water bath over 1 hour. Then, the obtained solution was stirred at room temperature for 2 days. The reaction mixture was gradually poured into 500 ml of water to produce a precipitate. The precipitate was filtrated and dissolved in chloroform. The chloroform phase was washed with saturated sodium bicarbonate water, and also washed twice with an aqueous 10% NaCl solution. The washed precipitate was dehydrated over magnesium sulfate anhydride, and then chloroform was distilled off under reduced pressure to obtain oxadiazole-N-oxide (2) (yield amount: 30.5 g, yield: 82%).

### (2) Synthesis of a Diketone Derivative (3)

In a 500 ml three-neck flask, 14.7 g (0.05 mol) of the oxadiazole-N-oxide (2) was dissolved in 400 ml of acetonitrile. To this, 6.0 g of metal zinc, 7 ml of acetic acid, and 20 ml of acetic acid anhydride were added. The obtained solution was cooled in a water bath so that the reaction temperature did not exceed 35° C. The mixture was stirred for 6 hours to terminate the reaction. The reaction mixture was filtrated to remove insoluble matters. Acetonitrile was distilled off under reduced pressure to obtain a residue. The residue was recrystallized from chloroform to obtain an oxadiazole dibenzoyl (3) (yield amount: 9.6 g, yield: 69%).

### (3) Synthesis of a Diphenyloxadiazolopyridine Ethyl Ester (4)

In a 500 ml three-neck flask, 10.0 g (0.035 mol) of the oxadiazole dibenzoyl (3) was dissolved in 300 ml of butanol. To this, 32.0 g (0.23 mol) of glycine ethyl ester hydrochloride was added. The obtained mixture was refluxed under heating for 24 hours. Butanol was distilled off under reduced pressure to obtain a residue. The residue was dissolved in 200 ml of chloroform and washed with 10% hydrochloric acid, then with saturated sodium bicarbonate water, and with an aqueous 10% NaCl solution. The washed residue was dried over magnesium sulfate anhydride to remove the solvent. The obtained residue was recrystallized from chloroform to obtain a 4,7-diphenyl-1,2,5-oxadiazolopyridine ethyl ester (4) (hereinafter referred to as an ester (4)) (yield amount: 7.6 g, yield: 65%).

### (4) Synthesis of an Active Ester (5)

In a 50 ml three-neck flask, 1.0 g (1.6 mmol) of the ester (4) of the Synthesis Example 1 was dissolved in 30 ml of ethanol. Then, 0.11 g (3.0 mmol) of KOH was added. After reflux under heating for 5 hours, the reaction mixture was added to 50 ml of water. The obtained aqueous solution was adjusted to pH 1 with hydrochloric acid to obtain a precipitate. The obtained precipitate was recrystallized from mixed solvent of water-ethanol (1:1) to obtain a carboxylic acid (yield amount: 0.47 g, yield: 81%).

In a 50 ml three-neck flask, 70 mg (0.17 mmol) of the carboxylic acid and 21 mg (0.18 mmol) of N-hydroxysuccinimide were dissolved in 20 ml of DMF. To this solution, 37 mg (0.17 mmol) of N,N′-dicyclohexylcarbodiimide dissolved in 5 ml of DMF was added dropwise over 30 minutes. After addition, the mixture was stirred at room temperature for 30 hours. DMF was distilled off under reduced pressure. The residue was isolated by silica column chromatography to obtain an active ester (5) (yield amount: 90 mg, yield: 78%).

(5) Reaction of Active Ester (5) with APS

The reaction between the active ester (5) and APS is shown below.

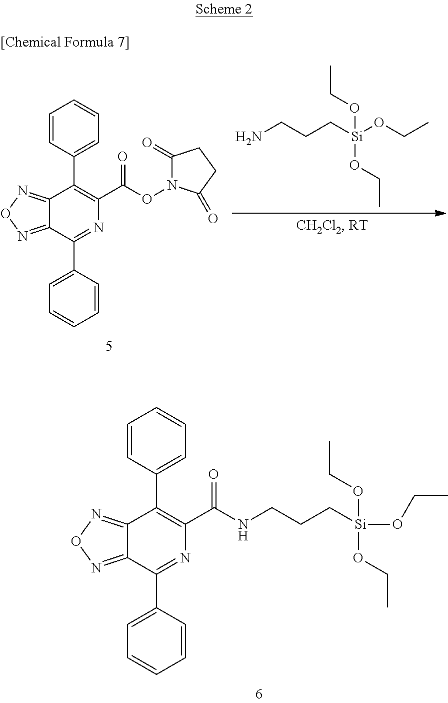 [SMILES: O=C(ON1C(=O)CCC1=O)c1nc(-c2ccccc2)c2nonc2c1-c1ccccc1 ; CCO[Si](CCCN)(OCC)OCC ; CCO[Si](CCCNC(=O)c1nc(-c2ccccc2)c2nonc2c1-c1ccccc1)(OCC)OCC]

In an eggplant flask, 100 mg (241 μmol) of the active ester (5) was placed and dissolved in 10 ml of dichloromethane. 0.1 ml (482 μmol) of APS was added into the flask, and reaction was started at room temperature. After reacting for 2 hours, the reaction solution was filtered through Celite and distilled off under reduced pressure with an evaporator. The product was purified by column chromatography treatment (CHCl₃:ACOAT=9.5:0.5), evaporated under reduced pressure, and dried with a vacuum pump to obtain APS-containing compound (6) (yield amount: 45.7 mg, yield: 36%).

Results of ¹H-NMR analysis for APS-containing compound (6) are shown below.

Based on the ¹H-NMR analysis, the signals at 8.670-8.650 ppm and 7.649-7.503 ppm were assigned to 2H and 8H hydrogen of benzene ring, respectively. The signal at 8.011 ppm was assigned to 1H hydrogen of amide bond. The signals at 3.849-3.800 ppm and 1.233-1.201 ppm were assigned to 6H and 9H hydrogen of triethoxysilane. The signals at 3.485-3.434 ppm, 1.777-1.701 ppm and 0.713-0.671 ppm were assigned to 2H in each of alkyl chains of triethoxysilane, in total 6H hydrogen of triethoxysilane.

### Synthetic Example 2

An example of synthesis of a nitrogen cation of 4,7-diphenyl-1,2,5-oxadiazolopyridine containing APS as an alkoxysilyl group will be described.

### (1) Synthesis of Active Ester (9)

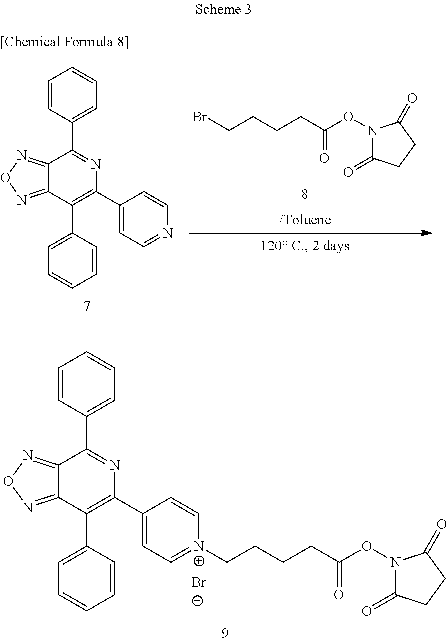 [SMILES: c1ccc(-c2nc(-c3ccncc3)c(-c3ccccc3)c3nonc23)cc1 ; O=C(CCCCBr)ON1C(=O)CCC1=O ; O=C(CCCC[n+]1ccc(-c2nc(-c3ccccc3)c3nonc3c2-c2ccccc2)cc1)ON1C(=O)CCC1=O ; [Br-]]

In a 20 ml two-necked flask, 0.2 g (2.85×10⁻⁴ mol) of pyridine compound (7) and 0.734 g (1.43×10⁻³ mol) of succinimidyl ester (8) were mixed. Then, the atmosphere of the flask was replaced with argon and deaerated. Then, 10 ml of toluene was added using a syringe and the mixture was stirred at 120° C. for 2 days. After cooling, the precipitate was filtered to obtain an active ester (9) (yield: 79%).

(2) Reaction of Active Ester (9) with APS

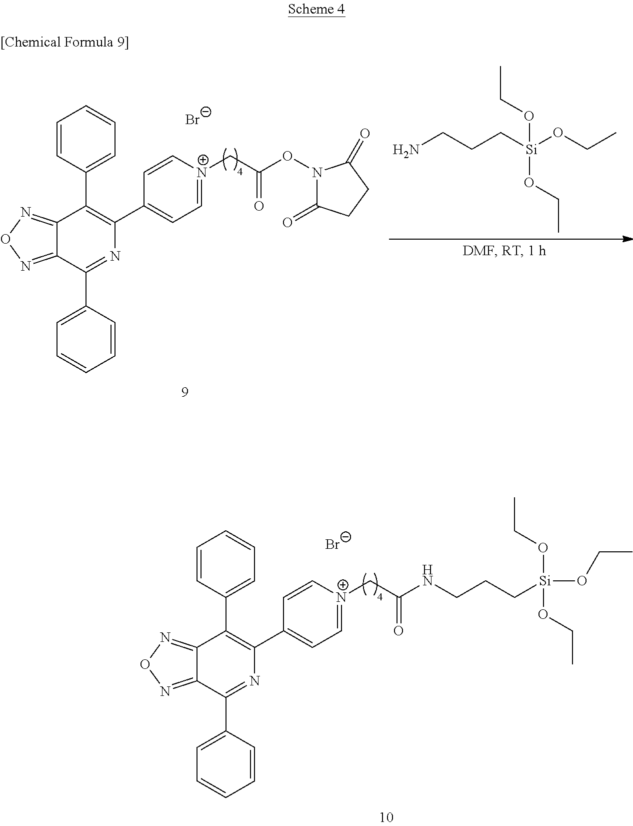

In a 5 ml eggplant flask, 14 mg (22.2 μmol) of the active ester (9) was dissolved in 0.7 ml of DMF containing molecular sieve 4 A. 0.7 ml of DMF and 5.2 μl (22.2 μmol) of APS were added to the reaction solution, and reaction was started at room temperature. After reacting for 1 hour, the reaction solution was distilled off under reduced pressure with an evaporator and dried with a vacuum pump to obtain APS-containing compound (10) (yield amount: 7.6 mg, yield: 46%).

Results of ¹H-NMR analysis (model JNM-KA400 manufactured by JEOL) for APS-containing compound (10) are shown below.

Based on the ¹H-NMR analysis, the signals at 9.365-9.348 ppm and 8.177-8.162 ppm were assigned to 4H hydrogen of pyridine ring. The signal at 8.740-8.716 ppm and 7.674-7.391 ppm were assigned to 10H hydrogen of right and left benzene rings, and the signal at 8.013 ppm was assigned to 1H hydrogen of amide bond. The signals at 5.045-5.008 ppm, 2.470-2.436 ppm, 2.163-2.1285 ppm, 1.801-1.767 ppm were assigned to 2H in each of alkyl chains beside the pyridine ring, in total 8H hydrogen. The signals at 3.822-3.690 ppm and 1.252-1.154 ppm were assigned to 6H and 9H hydrogen of triethoxysilane, respectively, and the signals at 3.222-3.173 ppm, 1.662-1.584 ppm and 0.647-0.605 ppm were assigned to 2H in each of alkyl chains beside amide bond, in total 6H hydrogen.

### Synthetic Example 3

An example of synthesis of an ester of 4,7-diphenyl-1,2,5-thiadiazolopyridine containing APS as an alkoxysilyl group will be described. The reaction scheme is shown below.

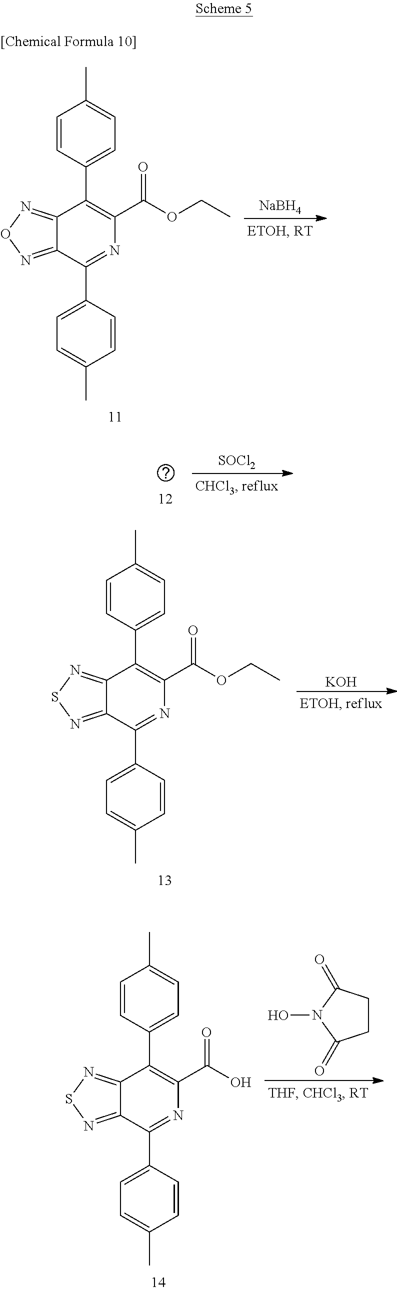

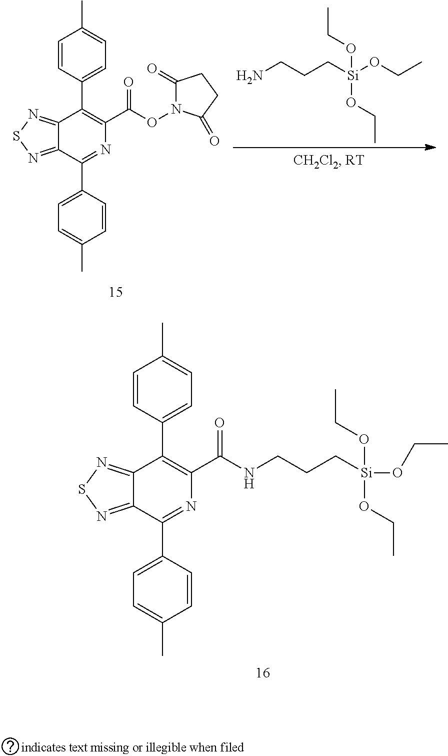 [SMILES: Cc1ccc(-c2nc(C(=O)ON3C(=O)CCC3=O)c(-c3ccc(C)cc3)c3nsnc23)cc1 ; CCO[Si](CCCN)(OCC)OCC ; CCO[Si](CCCNC(=O)c1nc(-c2ccc(C)cc2)c2nsnc2c1-c1ccc(C)cc1)(OCC)OCC]

### (1) Synthesis of Diamine Compound

In an eggplant flask, 383 mg (905 μmol) of ethyl ester (10) was dissolved in 30 ml of ethanol. 249 mg (6.60 mmol) of sodium borohydride was added, and reaction was started at room temperature. After reacting for 2 hours, the reaction solution was poured into water and extracted with a chloroform solvent (30 ml at a time is supplied two times). Magnesium sulfate was added to the chloroform phase, and the chloroform phase was subjected to suction filtration and then the solvent was removed under reduced pressure. The product was purified by column chromatography treatment (silica gel 60N manufactured by Kanto Chemicals Co., CHCl₃ 100%) to obtain diamine compound (12) (yield amount: 70 mg, yield: 21%).

### (2) Synthesis of Ethyl Ester

In an eggplant flask, 70 mg (193 μmol) of diamine compound (12) was dissolved in 2 ml of chloroform. A mixed solution containing 1 ml (1.4 mmol) of SOCl₂ and 1 ml of chloroform were added to the flask, and reaction was started by reflux under heating in an oil bath. After reacting for 2 hours, the reaction solution was poured into water and extracted with a chloroform solvent (30 ml at a time is supplied two times). Magnesium sulfate was added to the chloroform phase, and the chloroform phase was subjected to suction filtration and then the solvent was removed under reduced pressure. The residue was washed with ethanol to obtain ethyl ester (13) (yield amount: 30 mg, yield: 40%).

### (3) Synthesis of Carboxylic Acid Compound

In an eggplant flask, 180 mg (462 μmol) of ethyl ester (13) was dissolved in 20 ml of ethanol with an oil bath set at 80° C. An aqueous solution containing 64 mg (1.15 mmol) of potassium hydroxide dissolved in 5 ml of water was added, and reaction was started. After reacting for 4 hours, the reaction solution was poured into water, and hydrochloric acid was slowly added until the pH of the reaction solution was changed to equal to or less than 1 while stirring at room temperature. The precipitated solid was subjected to suction filtration and then dried to obtain a carboxylic acid compound (14) (yield amount: 106 mg, yield: 63%).

### (4) Synthesis of Active Ester

In an eggplant flask, 100 mg (276 μmol) of carboxylic acid compound (14) and 34 mg (303 μmol) of N-hydroxysuccinimide were dissolved in 10 ml of mixed solvent of THF and CHCl₃ (THF:CHCl₃=4:1) at room temperature. A solution containing WSCI dissolved in 10 ml of mixed solvent of THF and CHCl₃ (THF:CHCl₃=1:4) was slowly dropped into the reaction solution, and the reaction was started. After dropping, reaction was continued for 4 hours. After the reaction, the reaction solution was evaporated under reduced pressure, and the residue was dissolved in chloroform and washed twice with an aqueous solution of sodium chloride. Magnesium sulfate was added to this solution, and this solution was subjected to filtration, and the solvent was removed under reduced pressure and dried with vacuum pump. The product was purified by column chromatography treatment (silica gel 60N manufactured by Kanto Chemicals Co., CHCl₃:ACOET=9:1) to obtain active ester (15) (yield amount: 89 mg, yield: 70%).

### (5) Synthesis of APS-Containing Compound

In an eggplant flask, 81 mg (176 μmol) of carboxylic acid compound (14) was dissolved in 10 ml of dichloromethane. To the reaction solution, 0.044 ml (193 μmol) of APS was added, and the reaction was started. After reacting for 2.5 hours, the reaction solution was distilled off under reduced pressure using an evaporator. The product was purified by column chromatography treatment (silica gel 60N manufactured by Kanto Chemicals Co., CHCl₃:ACOET-9.8:0.2) to obtain APS-containing compound (16) (yield amount: 59 mg, yield: 59%).

Results of ¹H-NMR analysis for APS-containing compound (16) are shown below.

Based on the ¹H-NMR analysis, the signals at 8.528-8.513 ppm and 7.410-7.339 ppm were assigned to 2H and 8H hydrogen of benzene ring, respectively. The signal at 8.274 ppm was assigned to 1H hydrogen of amide bond. The signals at 3.832-3.817 ppm and 1.220-1.203 ppm were assigned to 6H and 9H hydrogen of triethoxysilane. The signals at 3.444 ppm, 1.738 ppm and 0.698 ppm were assigned to 6H of alkyl chains of triethoxysilane. The signal at 2.502-2.448 ppm was assigned to 6H of upper and lower methyl groups of benzene.

### Synthetic Example 4

An example of synthesis of a nitrogen cation compound (containing vinyl group) of 4,7-diphenyl-1,2,5-thiadiazolopyridine containing APS as an alkoxysilyl group will be described. First, a synthesis of the nitrogen cation compound (containing vinyl group) will be described.

A reduction reaction of eater (4) synthesized in Synthetic Example 1 was carried out in the presence of NaBH₄ to obtain a diamino alcohol compound (5), and the diamino alcohol compound (5) was reacted with thionyl chloride to obtain a thia diazolopyridine chloromethyl compound (6), and the thia diazolopyridine chloromethyl compound (6) was reacted with triphenylphosphine to obtain a phosphonium salt (7), and further subjecting to Wittig reaction to obtain a vinyl compound (8), and a pyridinium salt (9) containing an active ester (including —CH═CH—) was synthesized. The reaction examples are shown below.

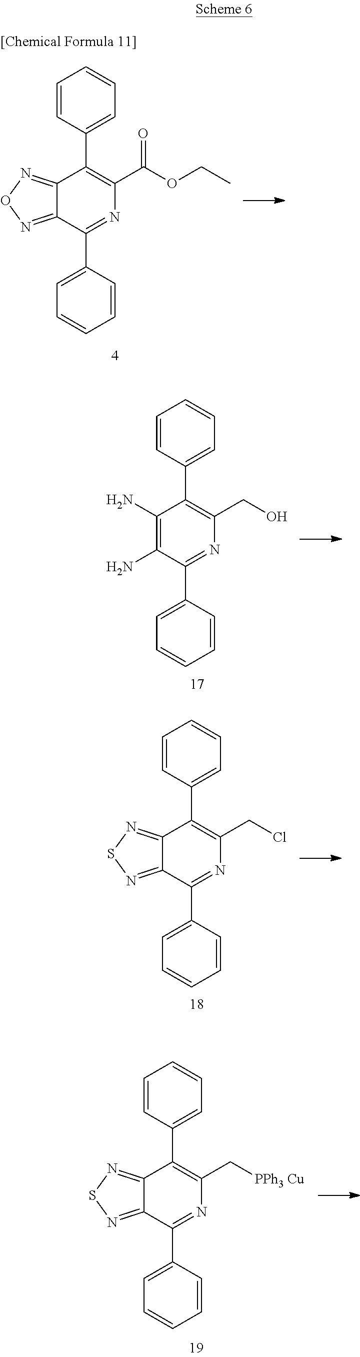

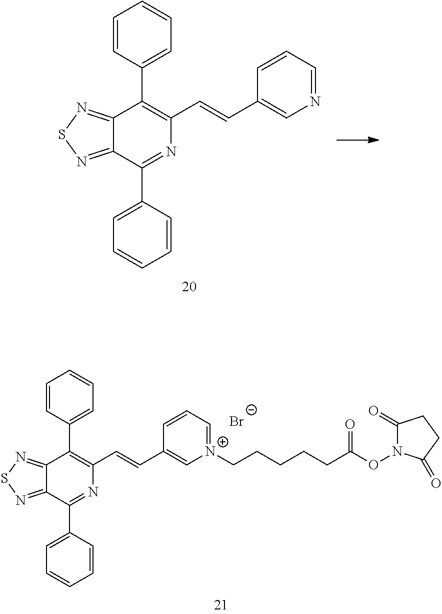 [SMILES: C(=C\c1nc(-c2ccccc2)c2nsnc2c1-c1ccccc1)/c1cccnc1 ; O=C(CCCCC[n+]1cccc(/C=C/c2nc(-c3ccccc3)c3nsnc3c2-c2ccccc2)c1)ON1C(=O)CCC1=O ; [Br-]]

### (1) Synthesis of a Diamino Alcohol Compound (17)

100 ml of ethanol solution containing 1.73 g (5 mmol) of ester (4) and 1.30 g (35 mmol) of NaBH₄ was refluxed under heating for 12 hours, and the reaction solution was poured into water and allowed to stand overnight, and the precipitate was filtered to obtain a diamino alcohol compound (17) (yield amount: 1.17 g, yield: 80%).

### (2) Synthesis of a Chloromethyl Compound (18)

A solution (3 ml) of the pyridine-NaBH₄ and thionyl chloride (6 ml) were added dropwise to 60 ml of chloroform solution containing diamino alcohol compound (17) (1.17 g), and the reaction solution was refluxed under heating for 3 hours and 30 minutes. Then, the reaction solution was poured into water, which was neutralized with saturated sodium bicarbonate water and then extracted with chloroform. The extract was dried over magnesium sulfate anhydride and the residue obtained after distillation under reduced pressure was subjected to column treatment (KantoC-60, Hexane/Chloroform=5/1 (v/v)) to obtain a chloromethyl (15) (yield amount: 1.11 g, yield: 82%).

### (3) Synthesis of a Phosphonium Salt (19)

A toluene solution (5 ml) containing 112.6 mg (0.33 mmol) of the chloromethyl compound (18) and 96 mg (0.37 mmol) of triphenylphosphine was refluxed under heating for 3 days, and the precipitate was filtrated to obtain a phosphonium salt (19) (yield amount: 108 mg, yield: 55%).

### (4) Synthesis of a Vinyl Compound (20)

The phosphonium salt (19) (140.5 mg, 0.23 mmol) was added to an ethanol solution (1 ml) containing m-formylpyridine (16 μL, 0.18 mmol) and potassium hydroxide (purity: 85%, 15 mg) under ice-cooling, and the mixture was stirred at the temperature for one hour and 30 minutes. The precipitate was filtrated and washed with ethanol and water and dried to obtain 4,7-diphenyl-1,2,5-oxadiazolopyridine-6-(4-vinylpyridine) (hereinafter referred to as a vinyl compound (20)) (yield amount: 44 mg, yield: 62%).

### (5) Synthesis of a Pyridinium Salt (21) Containing an Active Ester Compound

A toluene solution (2 ml) of the vinyl compound (20) (40 mg, 0.10 mmol) and a bromohexanoic acid active ester (32 mg, 0.11 mol) in toluene was refluxed under heating for 5 days, and then the precipitate was filtrated to obtain a pyridinium salt (21) containing an active ester.

### (6) Synthesis of APS-Containing Compound (22)

An reaction example is shown below.

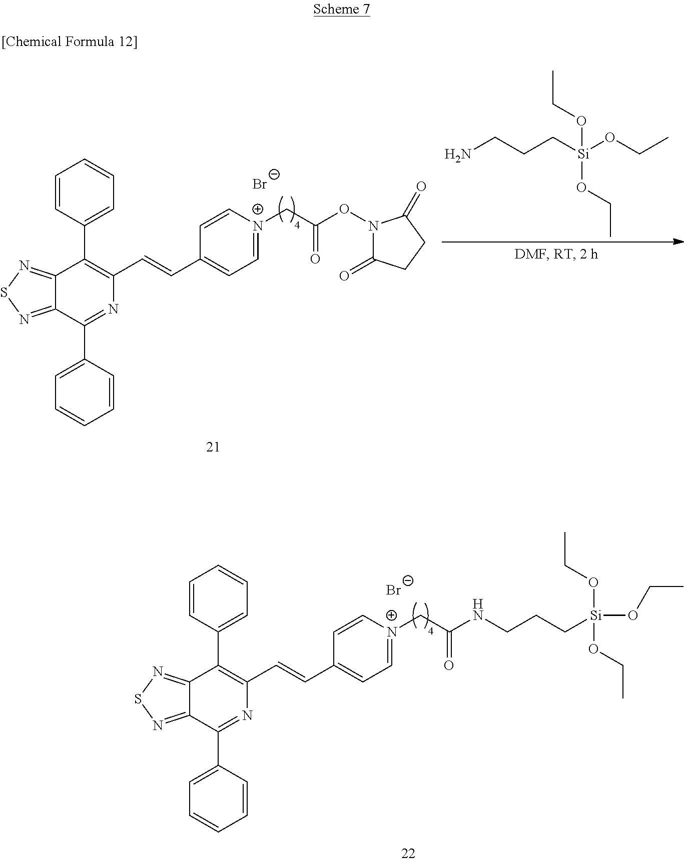

In an eggplant flask, 14 mg (20.8 μmol) of a pyridinium salt (21) containing an active ester was dissolved in 1.4 ml of DMF containing molecular sieve 4 A. To the reaction solution, 4.8 μl (20.8 μmol) of APS was added, and the reaction was started. After reacting for 2 hours, the reaction solution was distilled off under reduced pressure using an evaporator and dried with a vacuum pump to obtain APS-containing compound (22) (yield amount: 10.3 mg, yield: 64%).

The structure of the APS-containing compound (22) was confirmed by ¹H-NMR analysis.

### Synthetic Example 5

An example of synthesis of a thioether compound of 4,7-diphenyl-1,2,5-thiadiazolopyridine containing 3-mercaptopropyltrimethoxysilane (hereinafter abbreviated as MPS) as an alkoxysilyl group will be described. The reaction is shown below.

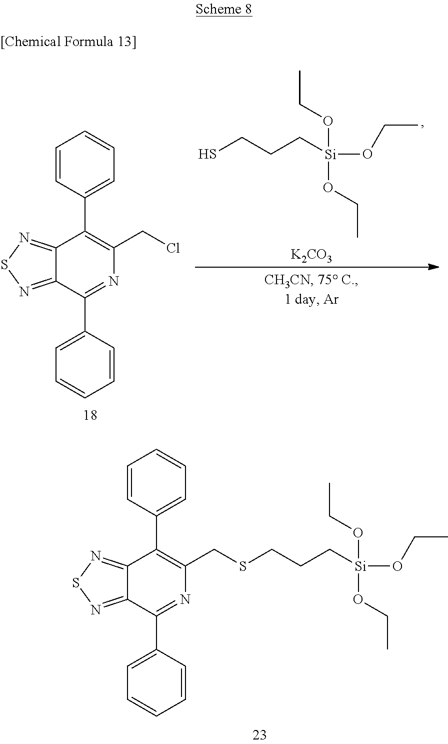 [SMILES: ClCc1nc(-c2ccccc2)c2nsnc2c1-c1ccccc1 ; CCO[Si](CCCS)(OCC)OCC ; "K2CO3" ; CCO[Si](CCCSCc1nc(-c2ccccc2)c2nsnc2c1-c1ccccc1)(OCC)OCC]

In a 50 ml of two-necked flask, 0.2 g (0.592 mmol) of chloromethyl compound (18) and 0.05 g (0.355 mmol, molar ratio 0.6) of potassium carbonate and 0.14 ml (0.592 mmol, molar ratio 1.0) of 3-mercaptopropyltriethoxysilane (hereinafter abbreviated as MPS) were placed, and the atmosphere of the flask was replaced with argon while reducing the pressure. Then, 20 ml of acetonitrile was added to this reaction solution using a glass syringe, and the reaction was carried out for 24 hours in an oil bath set at 75° C. The progress of the reaction was confirmed by TLC (CHCl₃:hexane=3:2), then the reaction solution was cooled to room temperature and further subjected to suction filtration, distillation under reduced pressure and vacuum drying. This product was subjected to silica gel column chromatography purification (Kanto 60N, CHCl₃:hexane=3:2). It was found that the product is impure as a result of ¹H-NMR analysis. Thus, purification by silica gel column chromatography (Kanto 60N, CHCl₃:hexane=3:2) was carried out again to obtain the target MPS-containing compound (23) (yield amount: 0.1795 g, yield: 56%).

Based on the ¹H-NMR analysis, the signal at 8.696-8.680 ppm was assigned to 2H hydrogen of phenyl group, which is due to the influence of the nitrogen of the oxadiazolopyridine skeleton, and the signal at 7.616-7.570 ppm was assigned to remaining 8H hydrogen of phenyl group. The signal at 4.000 ppm was assigned to 2H hydrogen between thiadiazolopyridine skeleton and thioether group, and the signals at 3.806-3.763 ppm and 1.205-1.181 ppm were assigned to 6H and 9H hydrogen of triethoxysilane, and the signals at 2.735-2.710 ppm, 1.736-1.684 ppm and 0.723-0.710 ppm were assigned to 2H hydrogen in each of alkyl chains.

### Synthetic Example 6

The reaction between the active ester compound (5) and MPS is shown below.

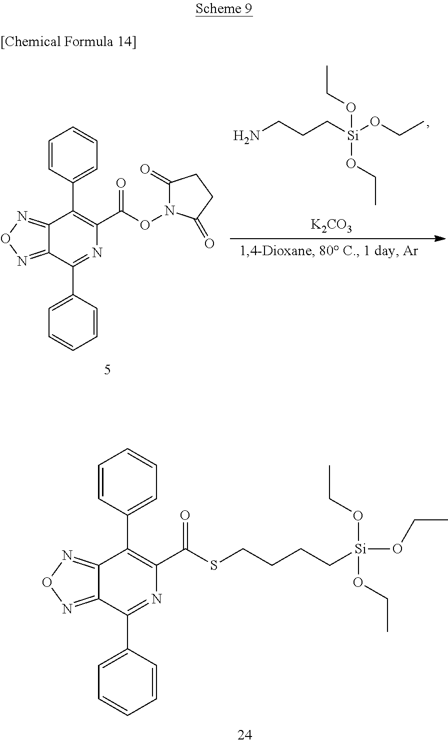 [SMILES: O=C(ON1C(=O)CCC1=O)c1nc(-c2ccccc2)c2nonc2c1-c1ccccc1 ; CCO[Si](CCCN)(OCC)OCC ; "K2CO3" ; CCO[Si](CCCCSC(=O)c1nc(-c2ccccc2)c2nonc2c1-c1ccccc1)(OCC)OCC]

In a 50 ml of eggplant flask, 0.1 g (0.725 mmol, molar ratio 1.5) of potassium carbonate and 0.12 ml (0.483 mmol, molar ratio 1.1) of 3-mercaptopropyltriethoxysilane (hereinafter abbreviated as MPS) were placed and stirred at room temperature with 10 ml of 1, 4-dioxane under argon atmosphere. To this, 0.02 g (0.483 mmol, molar ratio 1.1) of the active ester compound (5) dissolved in 15 ml of 1, 4-dioxane was added dropwise. After the dropwise addition, the reaction was carried out for 24 hours in an oil bath set at 80° C. (under argon atmosphere). The progress of the reaction was confirmed by TLC (CHCl₃ 100%), then the reaction solution was cooled to room temperature and further subjected to suction filtration, distillation under reduced pressure and vacuum drying. The product was subjected to silica gel column chromatography purification (Kanto 60N, CHCl₃ 100%). The product was subjected to ¹H-NMR analysis and was obtained the target MPS-containing compound (24) (yield amount: 0.16 g, yield: 62%).

Based on the ¹H-NMR analysis, the signal at 8.817-8.801 ppm was assigned to 2H hydrogen of phenyl group, which is due to the influence of the nitrogen of the oxadiazolopyridine skeleton, and the signal at 7.656-7.511 ppm was assigned to remaining 8H hydrogen of phenyl group. The signals at 3.835-3.800 ppm and 1.234-1.210 ppm were assigned to 6H and 9H hydrogen of triethoxysilane, and the signals at 3.021-2.996 ppm, 1.800-1.748 ppm and 0.776-0.748 ppm were assigned to 2H hydrogen in each of alkyl chains.

### Synthetic Example 7

An example of synthesis of 4,7-di[(1-naphthyl)thienyl]-1,2,5-oxadiazolopyridine-6-(4-pyridinium) containing APS as an alkoxysilyl group will be described. First, a synthesis of an active ester compound of 4,7-di[(1-naphthyl)thienyl]-1,2,5-oxadiazolopyridine-6-(4-pyridinium) will be described.

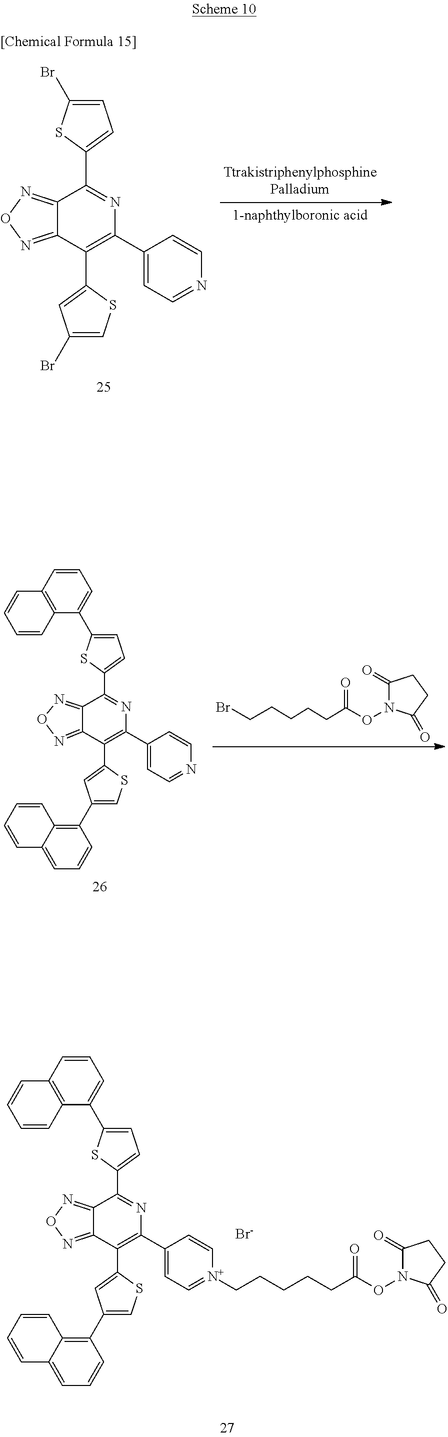 [SMILES: Brc1csc(-c2c(-c3ccncc3)nc(-c3ccc(Br)s3)c3nonc23)c1 ; c1ccc2c(-c3csc(-c4c(-c5ccncc5)nc(-c5ccc(-c6cccc7ccccc67)s5)c5nonc45)c3)cccc2c1 ; O=C(CCCCCBr)ON1C(=O)CCC1=O ; O=C(CCCCC[n+]1ccc(-c2nc(-c3ccc(-c4cccc5ccccc45)s3)c3nonc3c2-c2cc(-c3cccc4ccccc34)cs2)cc1)ON1C(=O)CCC1=O ; [Br-]]

### (1) Synthesis of a Pyridyl Compound (26) Using Suzuki Coupling

In an eggplant flask in which the atmosphere was replaced with argon, 200 mg (0.38 mmol) of the pyridyl compound (25) and 12.7 mg of tetrakis triphenylphosphine palladium were placed and dissolved in 2.8 ml of an aqueous 2M-sodium carbonate solution and 4 ml of benzene. In 2 ml of ethanol, 144 mg (0.83 mmol) of 1-naphthylboronic acid was dissolved, and the obtained mixture was poured into the reaction solution. Thereafter, the reaction solution was refluxed under heating at 80° C. for 6 hours. Into the reaction solution, 20 ml of water was poured for extraction using chloroform. Chloroform was distilled off under reduced pressure and the residue was recrystallized from hexane-chloroform. A pyridyl compound (26) was thus obtained in a yield amount of 190 mg and in a yield of 55%.

### (2) Synthesis of an Active Ester Compound (27)

In 8 ml of toluene, 300 mg (0.58 mmol) of the pyridyl compound (26) and 170 mg (0.58 mmol) of a bromohexanoic acid active ester were dissolved, and the obtained mixture was then stirred at room temperature overnight. After the completion of the reaction, the reaction mixture was subjected to suction filtration and the filtrated product was dried under vacuum to obtain an active ester compound (27).

The reaction between the active ester compound (27) and APS is shown below.

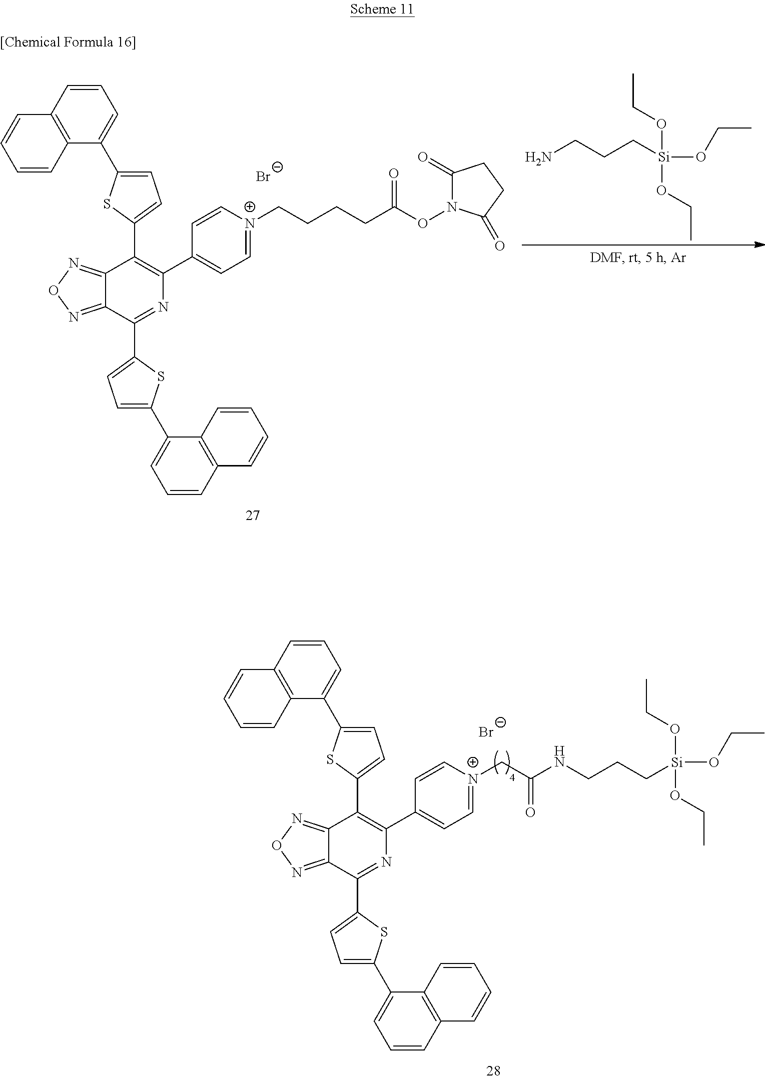

In a 50 ml of eggplant flask, 0.2 g (0.224 mmol, molar ratio 1.0) of compound (27) and 0.053 ml (0.224 mmol, molar ratio 1.0) of 3-aminopropyltriethoxysilane were placed and stirred for 5 hours at room temperature with 15 ml of DMF under argon atmosphere. After the completion of the reaction, the reaction solution was distilled off under reduced pressure using an evaporator and dried in vacuum. The product was subjected to silica gel column chromatography purification (Kanto 60N, CHCl₃:methanol-8:2). The product was subjected to ¹H-NMR analysis and was obtained the target APS-containing compound (28) (yield amount: 0.11 g, yield: 49%).

### Synthetic Example 8

An example of synthesis of 4,7-di[(2-naphthyl)thienyl]-1,2,5-oxadiazolopyridine-6-(4-pyridinium) containing APS as an alkoxysilyl group will be described. First, a synthesis of an active ester compound of 4,7-di[(2-naphthyl)thienyl]-1,2,5-oxadiazolopyridine-6-(4-pyridinium) will be described.

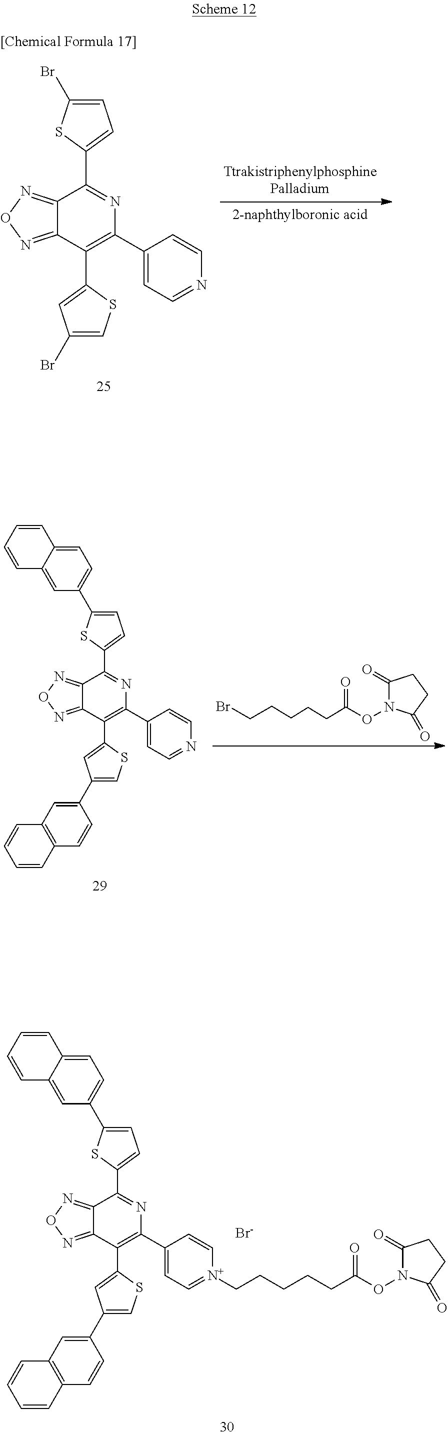 [SMILES: Brc1csc(-c2c(-c3ccncc3)nc(-c3ccc(Br)s3)c3nonc23)c1 ; c1ccc2cc(-c3csc(-c4c(-c5ccncc5)nc(-c5ccc(-c6ccc7ccccc7c6)s5)c5nonc45)c3)ccc2c1 ; O=C(CCCCCBr)ON1C(=O)CCC1=O ; O=C(CCCCC[n+]1ccc(-c2nc(-c3ccc(-c4ccc5ccccc5c4)s3)c3nonc3c2-c2cc(-c3ccc4ccccc4c3)cs2)cc1)ON1C(=O)CCC1=O ; [Br-]]

### (1) Synthesis of a Pyridyl Compound (29) Using Suzuki Coupling

In an eggplant flask in which the atmosphere was replaced with argon, 200 mg (0.38 mmol) of the pyridyl compound (25) and 12.7 mg of tetrakis triphenylphosphine palladium were placed and dissolved in 2.8 ml of an aqueous 2M-sodium carbonate solution and 4 ml of benzene. In 2 ml of ethanol, 144 mg (0.83 mmol) of 2-naphthylboronic acid was dissolved, and the obtained mixture was poured into the reaction solution. Thereafter, the reaction solution was refluxed under heating at 80° C. for 5 hours. Into the reaction solution, 15 ml of water was poured for extraction using chloroform. Chloroform was distilled off under reduced pressure and the residue was recrystallized from hexane-chloroform. A pyridyl compound (29) was thus obtained in a yield amount of 220 mg and in a yield of 64%.

### (2) Synthesis of an Active Ester Compound (30)

In 8 ml of toluene, 300 mg (0.58 mmol) of the pyridyl compound (29) and 170 mg (0.58 mmol) of a bromohexanoic acid active ester were dissolved, and the obtained mixture was then stirred at room temperature overnight. After the completion of the reaction, the reaction mixture was subjected to suction filtration and the filtrated product was dried under vacuum to obtain an active ester compound (30).

The reaction between the active ester compound (30) and APS is shown below.

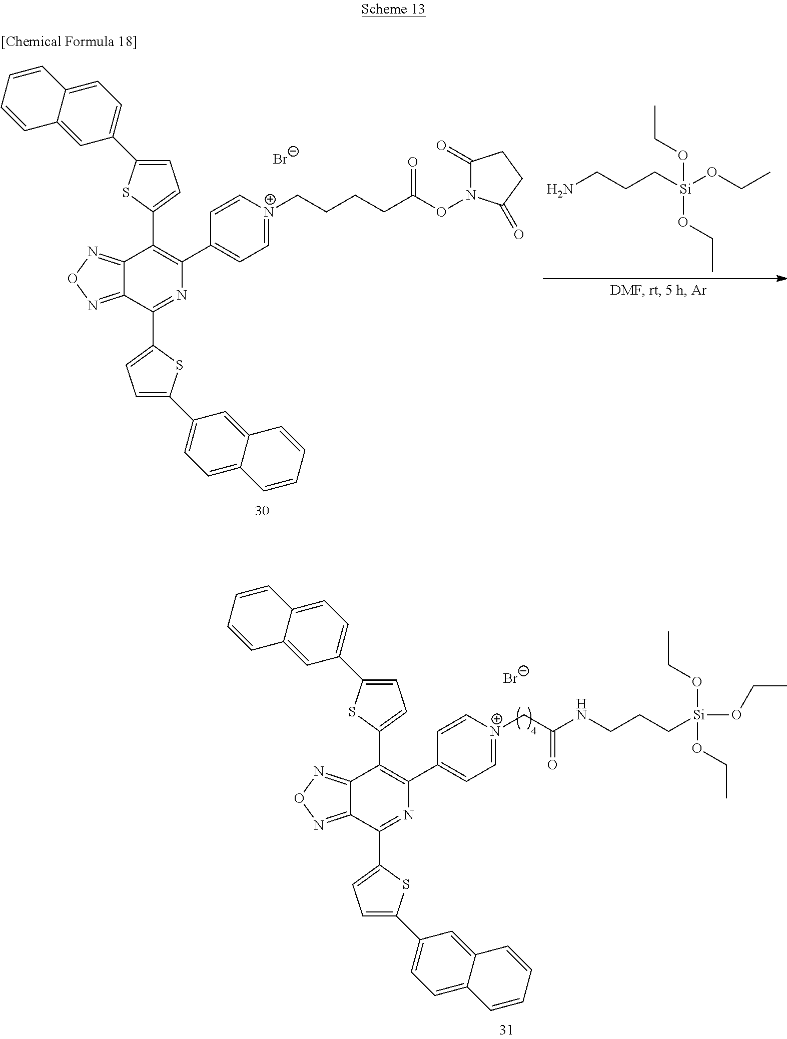

In a 50 ml of eggplant flask, 0.2 g (0.224 mmol, molar ratio 1.0) of the active ester compound (30) and 0.053 ml (0.224 mmol, molar ratio 1.0) of 3-aminopropyltriethoxysilane were placed and stirred for 5 hours at room temperature with 15 ml of DMF under argon atmosphere. After the completion of the reaction, the reaction solution was distilled off under reduced pressure using an evaporator and dried in vacuum. The product was subjected to silica gel column chromatography purification (Kanto 60N, CHCl₃:methanol=8:2). The product was subjected to ¹H-NMR analysis and was obtained the target MPS-containing compound ( ) (yield amount: 0.13 g, yield: 58%).

### Synthetic Example 9

An example of synthesis of 4,7-di[(2-biphenyl)thienyl]-1,2,5-oxadiazolopyridine-6-(4-pyridinium) containing APS as an alkoxysilyl group will be described. First, a synthesis of an active ester compound of 4,7-di[(2-biphenyl)thienyl]-1,2,5-oxadiazolopyridine-6-(4-pyridinium) will be described.

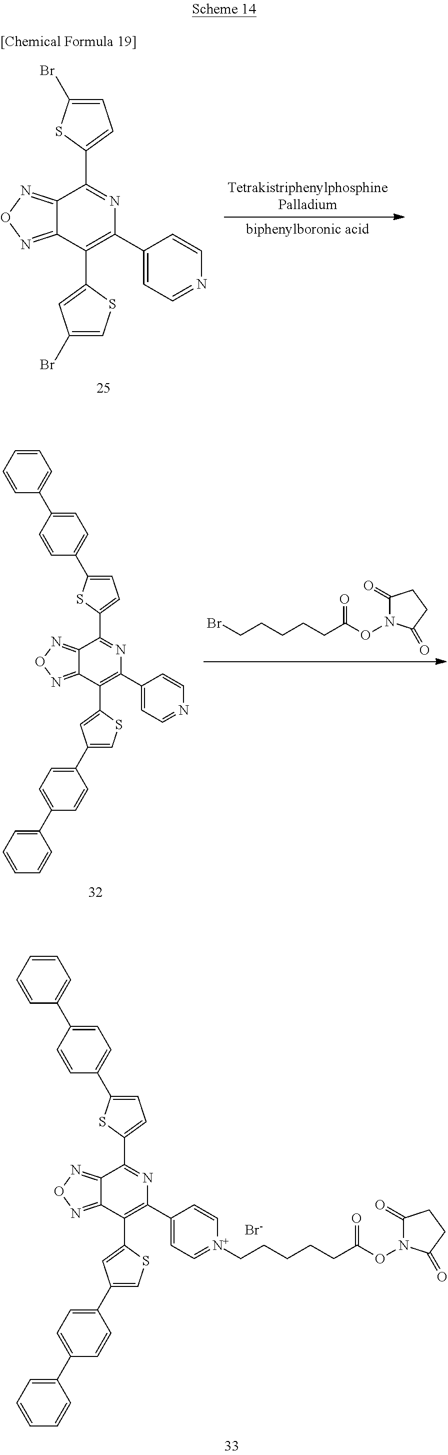 [SMILES: Brc1csc(-c2c(-c3ccncc3)nc(-c3ccc(Br)s3)c3nonc23)c1 ; c1ccc(-c2ccc(-c3csc(-c4c(-c5ccncc5)nc(-c5ccc(-c6ccc(-c7ccccc7)cc6)s5)c5nonc45)c3)cc2)cc1 ; O=C(CCCCCBr)ON1C(=O)CCC1=O ; O=C(CCCCC[n+]1ccc(-c2nc(-c3ccc(-c4ccc(-c5ccccc5)cc4)s3)c3nonc3c2-c2cc(-c3ccc(-c4ccccc4)cc3)cs2)cc1)ON1C(=O)CCC1=O ; [Br-]]

### (1) Synthesis of a Pyridyl Compound (32) Using Suzuki Coupling

In an eggplant flask in which the atmosphere was replaced with argon, 200 mg (0.38 mmol) of the pyridyl compound (25) and 12.7 mg of tetrakis triphenylphosphine palladium were placed and dissolved in 2.8 ml of an aqueous 2M-sodium carbonate solution and 4 ml of benzene. In 2 ml of ethanol, 164 mg (0.83 mmol) of biphenylboronic acid was dissolved, and the obtained mixture was poured into the reaction solution. Thereafter, the reaction solution was refluxed under heating at 80° C. for 5 hours. Into the reaction solution, 20 ml of water was poured for extraction using chloroform. Chloroform was distilled off under reduced pressure and the residue was recrystallized from hexane-chloroform. A pyridyl compound (32) was thus obtained in a yield amount of 155 mg and in a yield of 61%.

### (2) Synthesis of an Active Ester Compound (33)

In 8 ml of toluene, 300 mg (0.45 mmol) of the pyridyl compound (32) and 145 mg (0.49 mmol) of a bromohexanoic acid active ester were dissolved, and the obtained mixture was then stirred at room temperature overnight. After the completion of the reaction, the reaction mixture was subjected to suction filtration and the filtrated product was dried under vacuum to obtain an active ester compound (33).

The reaction between the active ester compound (33) and APS is shown below.

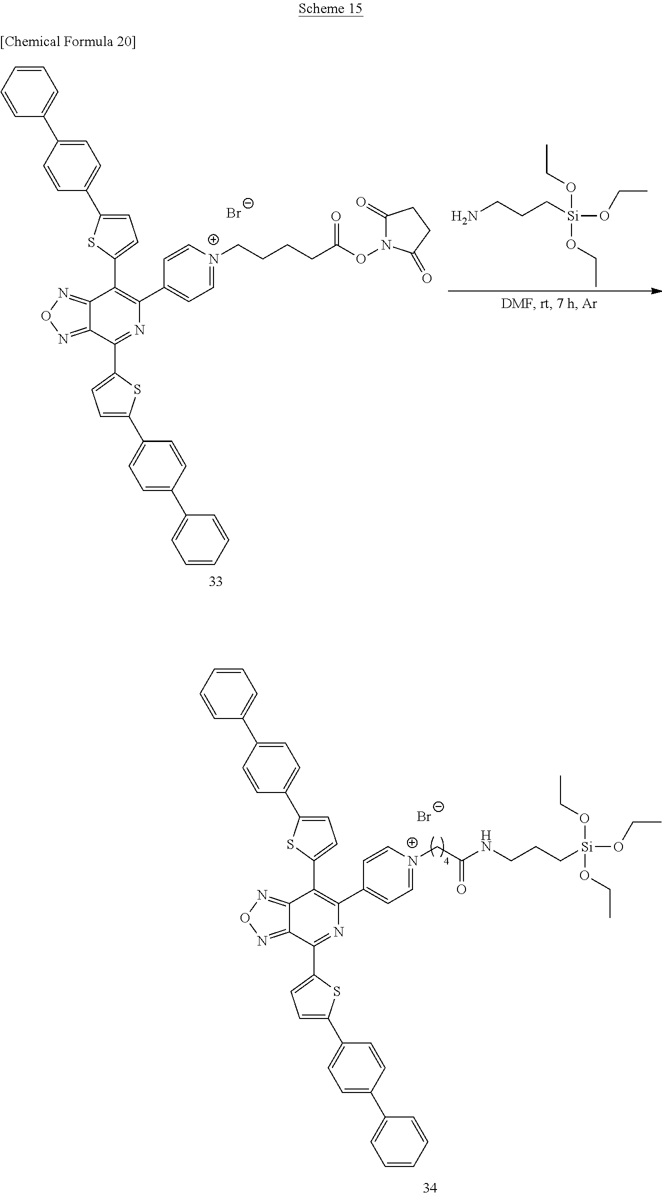

In a 50 ml of eggplant flask, 0.2 g (0.213 mmol, molar ratio 1.0) of the active ester compound (33) and 0.05 ml (0.213 mmol, molar ratio 1.0) of 3-aminopropyltriethoxysilane were placed and stirred for 7 hours at room temperature with 15 ml of DMF under argon atmosphere. After the completion of the reaction, the reaction solution was distilled off under reduced pressure using an evaporator and dried in vacuum. The product was subjected to silica gel column chromatography purification (Kanto 60N, CHCl₃:methanol=8:2). The product was subjected to ¹H-NMR analysis and was obtained the target APS-containing compound (34) (yield amount: 0.092 g, yield: 41%).

### Synthetic Example 10

An example of synthesis of thioether compound (36) containing MPS as an alkoxysilyl group will be described.

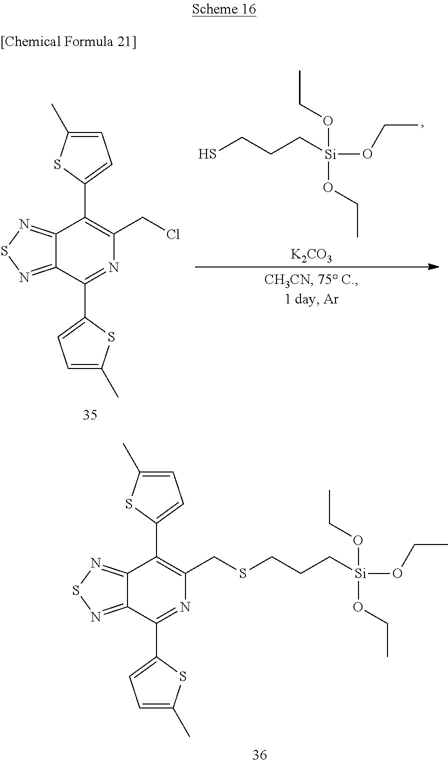

In a 50 ml of two-necked flask, 0.2 g (0.529 mmol, molar ratio 1.0) of chloro compound (35) and 0.073 g (0.529 mmol, molar ratio 1.0) of potassium carbonate and 0.13 ml (0.529 mmol, molar ratio 1.0) of 3-mercaptopropyltriethoxysilane were placed, and the atmosphere of the flask was replaced with argon while reducing the pressure. Then, 20 ml of acetonitrile was added to this reaction solution using a glass syringe, and the reaction was carried out for 24 hours in an oil bath set at 75° C. The progress of the reaction was confirmed by TLC (CHCl₃:hexane=3:2), then the reaction solution was cooled to room temperature and further subjected to suction filtration, distillation under reduced pressure and vacuum drying. This product was subjected to silica gel column chromatography purification (Kanto 60N, CHCl₃:hexane=3:2). It was found that the product is impure as a result of ¹H-NMR analysis. Thus, purification by silica gel column chromatography (Kanto 60N, CHCl₃ 100%) was carried out again to obtain the target thioether compound (36)(crude) (yield amount: 0.075 g, yield: 25%).

### Synthetic Example 11

An example of synthesis of thioester compound (38) containing MPS as an alkoxysilyl group will be described.

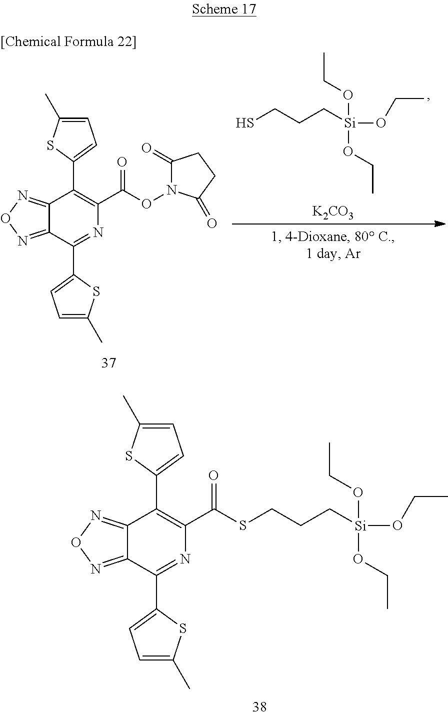 [SMILES: Cc1ccc(-c2nc(C(=O)ON3C(=O)CCC3=O)c(-c3ccc(C)s3)c3nonc23)s1 ; CCO[Si](CCCS)(OCC)OCC ; "K2CO3" ; CCO[Si](CCCSC(=O)c1nc(-c2ccc(C)s2)c2nonc2c1-c1ccc(C)s1)(OCC)OCC]

In a 50 ml of eggplant flask, 0.09 g (0.66 mmol, molar ratio 1.5) of potassium carbonate and 0.12 ml (0.484 mmol, molar ratio 1.1) of 3-mercaptopropyltriethoxysilane were placed and stirred at room temperature with 10 ml of 1,4-dioxane under argon atmosphere. To this, 0.02 g (0.44 mmol, molar ratio 1.0) of the active ester compound (37) dissolved in 15 ml of 1,4-dioxane was added dropwise. After the dropwise addition, the reaction was carried out for 24 hours in an oil bath set at 80° C. (under argon atmosphere). The progress of the reaction was confirmed by TLC (CHCl₃ 100%), then the reaction solution was cooled to room temperature and further subjected to suction filtration, distillation under reduced pressure and vacuum drying. The product was subjected to silica gel column chromatography purification (Kanto 60N, CHCl₃ 100%). The product was subjected to ¹H-NMR analysis and was obtained the target thioester compound (38) (yield amount: 0.1 g, yield: 39%).

### Synthetic Example 12

An example of synthesis of pyridinium salt (40) containing 3-iodopropyltrimethoxysilane as an alkoxysilyl group will be described.

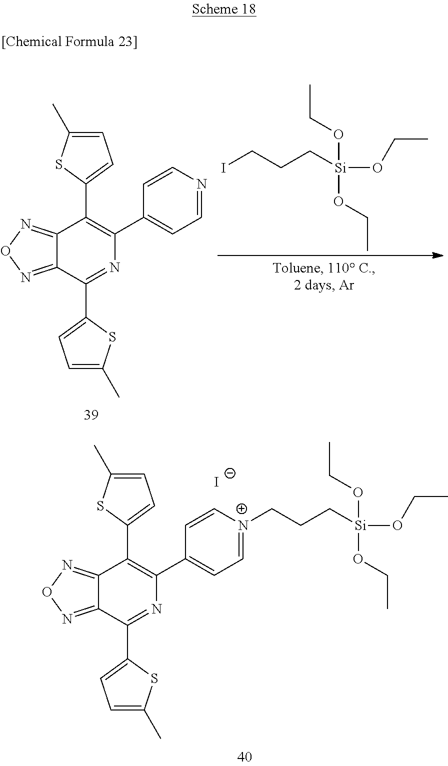

In a 30 ml of eggplant flask, 0.2 g (0.512 mmol, molar ratio 1.0) of pyridyl compound (39) and 0.85 g (2.56 mmol, molar ratio 5.0) of 3-iodopropyltriethoxysilane and 10 ml of toluene were placed, and the atmosphere of the flask was replaced with argon under reduced pressure while stirring at room temperature. Then, the reaction was carried out for 2 days in an oil bath set at 110° C. The progress of the reaction was confirmed by TLC (CHCl₃ 100%), then the reaction solution was cooled to room temperature and further subjected to distillation under reduced pressure and vacuum drying. The residue was washed with hexane and subjected to suction filtration and vacuum drying. The product was subjected to silica gel column chromatography purification (Kanto 60N, CHCl₃:methanol=8:2). The product was subjected to ¹H-NMR analysis and was obtained the target pyridinium salt (40) (yield amount: 0.11 g, yield: 30%).

### Synthetic Example 13

An example of synthesis of pyridinium salt (42) containing 3-iodopropyltrimethoxysilane as an alkoxysilyl group will be described.

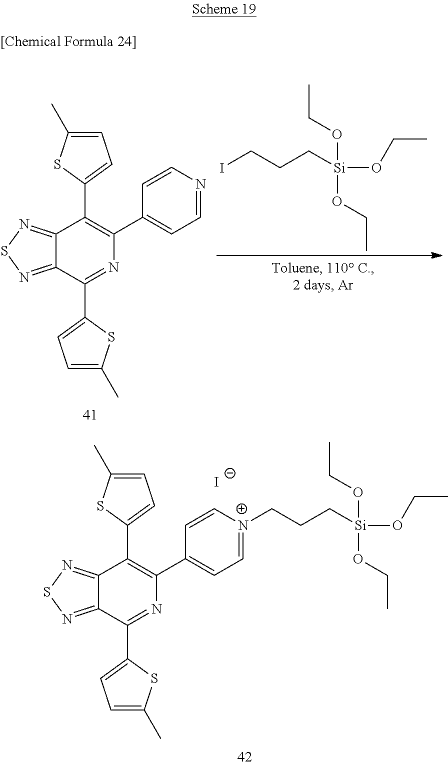

In a 30 ml of eggplant flask, 0.2 g (0.492 mmol, molar ratio 1.0) of pyridyl compound (41) and 0.82 g (2.46 mmol, molar ratio 5.0) of 3-iodopropyltriethoxysilane and 10 ml of toluene were placed, and the atmosphere of the flask was replaced with argon under reduced pressure while stirring at room temperature. Then, the reaction was carried out for 2 days in an oil bath set at 110° C. The progress of the reaction was confirmed by TLC (CHCl₃ 100%), then the reaction solution was cooled to room temperature and further subjected to distillation under reduced pressure and vacuum drying. The residue was washed with hexane and subjected to suction filtration and vacuum drying. The product was subjected to silica gel column chromatography purification (Kanto 60N, CHCl₃:methanol=8:2). The product was subjected to ¹H-NMR analysis and was obtained the target pyridinium salt (42) (yield amount: 0.12 g, yield: 33%).

### Evaluation of Fading Property

### Experiment 1

The alkoxyl group-containing EL dyes produced in Synthetic Examples 1-13 were dissolved in chloroform, and the solution was dropped on a slide glass and then subjected to drying to prepare membrane like samples. The ultraviolet rays were irradiated to the prepared samples using an ultraviolet lump (AS ONE SLUV-4:irradiation wavelength 365 nm), and after a lapse of a predetermined time, observation with the fluorescence microscope below was performed. The change in the fluorescence intensity before and after the irradiation was evaluated by visually observing the taken photograph.

### Microscope: OLYMPUS BX50

Excitation filter: ET405/40X
Dichroic mirror: T470pxr
absorption filter: ET545/70 m

### (Results)

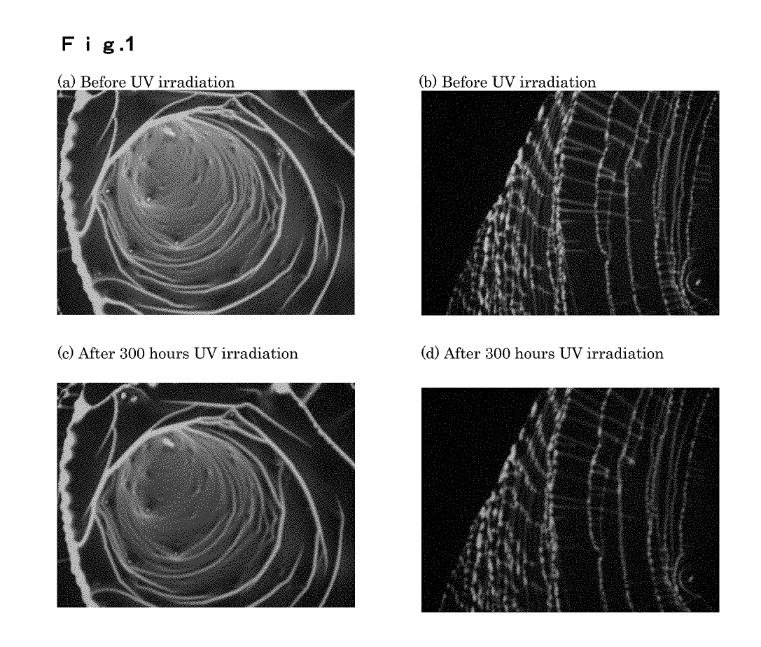

The results of absorption wavelength, fluorescence wavelength, and changes of fluorescence intensity after UV irradiation of 6 hours are shown in Table 1. Even after UV irradiation, the same fluorescence intensity as before UV irradiation was obtained. As for sample of the APS-containing compound (10) produced in Synthetic Example 2, FIG. 1 shows fluorescence micrographs taken before UV irradiation test and after 300 hours UV irradiation. Even after 300 hours UV irradiation, the same fluorescence intensity as before UV irradiation was obtained.

TABLE 1

| | | Absorp- | Fluores- | | Change of |
|---|---|---|---|---|---|
| | | tion | cence | | fluorescence |
| | Com- | wave- | wave- | Stokes | intensity |
| | pound | length | length | shift | after 60 hours |
| | No. | (nm) | (nm) | (nm) | UV irradiation |
| Synthetic | 6 | 382 | 519 | 137 | No change |
| Example | | | | | |
| 1 | | | | | |
| Synthetic | 10 | 391 | 528 | 137 | the same |
| Example | | | | | as above |
| 2 | | | | | |
| Synthetic | 16 | 399 | 540 | 141 | the same |
| Example | | | | | as above |
| 3 | | | | | |
| Synthetic | 22 | 394 | 531 | 137 | the same |
| Example | | | | | as above |
| 4 | | | | | |
| Synthetic | 23 | 384 | 513 | 129 | the same |
| Example | | | | | as above |
| 5 | | | | | |
| Synthetic | 24 | 381 | 544 | 163 | the same |
| Example | | | | | as above |
| 6 | | | | | |
| Synthetic | 28 | 495 | 675 | 180 | the same |
| Example | | | | | as above |
| 7 | | | | | |
| Synthetic | 31 | 521 | 698 | 177 | the same |
| Example | | | | | as above |
| 8 | | | | | |
| Synthetic | 34 | 509 | 681 | 172 | the same |
| Example | | | | | as above |
| 9 | | | | | |
| Synthetic | 36 | 448 | 640 | 192 | the same |
| Example | | | | | as above |
| 10 | | | | | |
| Synthetic | 38 | 458 | 661 | 203 | the same |
| Example | | | | | as above |
| 11 | | | | | |
| Synthetic | 40 | 464 | 665 | 201 | the same |
| Example | | | | | as above |
| 12 | | | | | |
| Synthetic | 42 | 456 | 653 | 197 | the same |
| Example | | | | | as above |
| 13 | | | | | |

The alkoxyl group-containing organic EL dyes produced in Synthetic Examples 1-13 all showed a Stokes shift of 100 nm or more, and in particular, those organic EL dyes produced in Synthetic Examples 7-13 having substituted thienyl group in R2 and R3 showed a Stokes shift of 170 nm or more. This enables the high sensitivity detection without being affected by the excitation light. Further, since those organic EL dyes produced in Synthetic Examples 7-13 have fluorescence wavelength in the near-infrared region of 640 nm or more, those organic EL dyes become an effective tool for detecting morphological change and functional change of living tissue, and are expected as a high sensitivity fluorescent reagent for biological imaging.

1-13. (canceled)

14. An alcoxysilyl group-containing organic EL dye represented by the general formula X—Y-Q-Z—Si(R₁)ₙ(OR₂)₃₋ₙ, wherein
X is an organic EL dye,
Y is a direct bond or —(CH₂)ₚ— or —(O—CH₂CH₂)_{q}—,
p represents an integer from 1 to 10,
q represents an integer from 1 to 10,
Q is a bond selected from the group consisting of amide bond, ether bond, thioether bond, thioester bond, thiourea bond, disulfide bond, and polyoxyethylene bond,
Z is —(CH₂)₃— or —(CH₂)₂NH(CH₂)₃—,
R₁ and R₂ represent an alkyl group having 1 to 7 carbon atoms,
n represents 0 or 1, and
wherein the organic EL dye is a diazoropyridine derivative or an imidazole derivative.

15. The alcoxysilyl group-containing organic EL dye according to claim 14, wherein the diazoropyridine derivative is represented by the following general formulae (1), (2), or (3), and wherein M₁ is directly bonded to the linking group, or indirectly bonded to the linking group through —(CH₂)ₚ—, wherein p represents an integer of 1 to 10, or —(O—CH₂CH₂)_{q}—, wherein q represents an integer of 1 to 10: 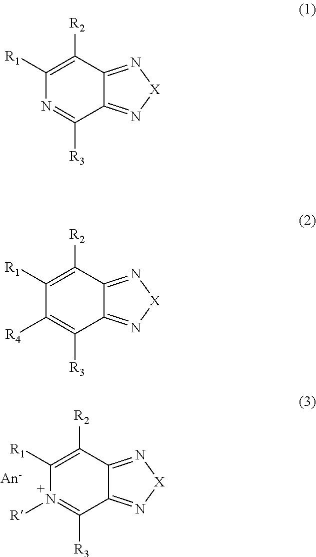
wherein
R₁ in the formulae (1) and (3) and one of R₁ and R₄ in the formula (2) are each represented by the general formula L₁-M₁,
M₁ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent,
L₁ represents a linker which is represented by —(CH═CR₆)ₛ— and which connects M₁ with a center pyridine ring or a center benzene ring,
s represents an integer of from 1 to 5,
R₆ represents any one of a hydrogen atom; a linear or branched alkyl group which may have a substituent and has 1 to 6 carbon atoms; a sulfo group which may have a substituent; a heterocyclic group selected from the group consisting of an imidazolium group, a pyridinium group, and a furan group, each of which may have a substituent; an amino group selected from the group consisting of a secondary amino group, a tertiary amino group, and a quaternary amino group, each of which may have a substituent; a hydroxy group which may have a substituent; an alkoxy group which may have a substituent; an aldehyde group which may have a substituent; a carboxyl group which may have a substituent; and an aromatic group which may have a substituent,
the residues of R₁ and R₄ in the formula (2) and R₂ and R₃ in the formulae (1) to (3) each independently represent a hydrogen atom, a halogen atom, or an aromatic hydrocarbon group, an aliphatic hydrocarbon group, or a heterocyclic group, each of which may have a substituent,
X represents a nitrogen atom, a sulfur atom, an oxygen atom, a selenium atom, or a boron atom, each of which may have a substituent,
R′ represents an aliphatic hydrocarbon group including an alkyl group which may have an aromatic ring or an aromatic hydrocarbon group, and
An⁻ represents a halide ion, CF₃SO₃⁻, BF₄⁻, or PF₆⁻.

16. The alcoxysilyl group-containing organic EL dye according to claim 15, wherein R₂ and R₃ represent each independently a thienyl group, a furanyl group, a pyrrolyl group, an imidazolyl group, an oxazolyl group, a thiadiazolyl group, a pyrazolyl group, a pyridyl group, or a quinolyl group, each of which may have a substituent.

17. The alcoxysilyl group-containing organic EL dye according to claim 16, wherein R₂ and R₃ each represent a thienyl group which may have a substituent, and wherein the substituent is an aromatic hydrocarbon group, an aliphatic hydrocarbon group, or a heterocyclic group, each of which may have a substituent.

18. The alcoxysilyl group-containing organic EL dye according to claim 14, wherein the diazoropyridine derivative is represented by the following general formulae (4), (5), or (6), and wherein M₂ is directly bonded to the linking group, or indirectly bonded to the linking group through —(CH₂)ₚ—, wherein p represents an integer of 1 to 10, or —(O—CH₂CH₂)_{q}—, wherein q represents an integer of 1 to 10: 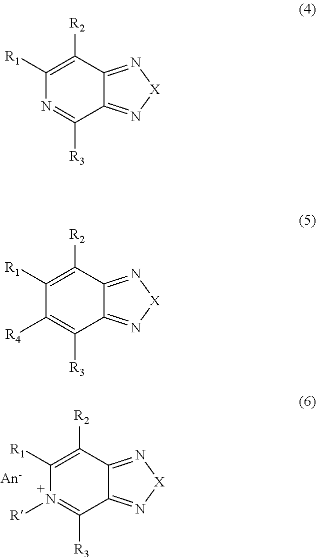
wherein
R₁ in the formulae (4) and (6) and one of R₁ and R₄ in the formula (5) are each represented by the general formula L₂-M₂,
M₂ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent, L₂ represents a linker which connects M₂ with a center pyridine ring or a center benzene ring and which represents a direct bond or one or more types of functional groups selected from the group consisting of —(CH₂)ₛ—, wherein s represents an integer of from 1 to 4, —NHCOO—, —CONH—, —CON(CH₃)—, —COO—, —SO₂NH—, —HN—C(═NH)—NH—, —O—, —S—, —NR, wherein R represents an alkyl group, —Ar—, wherein Ar represents an aromatic hydrocarbon group, and —CO—Ar—NR—, the residues of R₁ and R₄ in the formula (5) and R₂ and R₃ in the formulae (4) to (6) each independently represent a thienyl group, a furanyl group, a pyrrolyl group, an imidazolyl group, an oxazolyl group, a thiadiazolyl group, a pyrazolyl group, a pyridyl group, or a quinolyl group, each of which may have a substituent,
X represents a nitrogen atom, a sulfur atom, an oxygen atom, a selenium atom, or a boron atom, each of which may have a substituent,
R′ represents an aliphatic hydrocarbon group including an alkyl group which may have an aromatic ring or an aromatic hydrocarbon group, and
An⁻ represents a halide ion, CF₃SO₃⁻, BF₄⁻, or PF₆⁻.

19. The alcoxysilyl group-containing organic EL dye according to claim 18, wherein R₂ and R₃ represent each independently a thienyl group, a furanyl group, a pyrrolyl group, an imidazolyl group, an oxazolyl group, a thiadiazolyl group, a pyrazolyl group, a pyridyl group, or a quinolyl group, each of which may have a substituent.

20. The alcoxysilyl group-containing organic EL dye according to claim 19, wherein R₂ and R₃ each represent a thienyl group which may have a substituent, and wherein the substituent is an aromatic hydrocarbon group, an aliphatic hydrocarbon group, or a heterocyclic group, each of which may have a substituent.

21. The alcoxysilyl group-containing organic EL dye according to claim 14, wherein the diazoropyridine derivative is represented by the following general formulae (7), (8), or (9), and wherein R₁ or R₄ is directly bonded to the linking group: 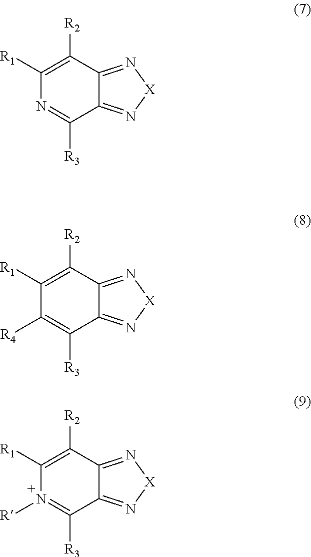
wherein
R₁, R₂, R₃ and R₄ in the formula (7), (8) and (9) each independently represent a hydrogen atom, a halogen atom, or an aromatic hydrocarbon group, or an aliphatic hydrocarbon group, or a heterocyclic group, each of the aromatic hydrocarbon group, the aliphatic hydrocarbon group and the heterocyclic group may have a substituent selected from the group consisting of an alkyl group, an alkenyl group, an alkynyl group, an alkoxy group, an alkyl ester group, a phosphate ester group, a sulfate ester group, a nitrile group, a hydroxyl group, a cyano group, a sulfonyl group, an aromatic hydrocarbon group, and a heterocyclic group,
X represents a nitrogen atom, a sulfur atom, an oxygen atom, a selenium atom, or a boron atom, each of which may have a substituent,
R′ represents an aliphatic hydrocarbon group including an alkyl group which may have an aromatic ring or an aromatic hydrocarbon group, and
An⁻ represents a halide ion, CF₃SO₃⁻, BF₄⁻, or PF₆⁻.

22. The alcoxysilyl group-containing organic EL dye according to claim 21, wherein R₂ and R₃ represent each independently a thienyl group, a furanyl group, a pyrrolyl group, an imidazolyl group, an oxazolyl group, a thiadiazolyl group, a pyrazolyl group, a pyridyl group, or a quinolyl group, each of which may have a substituent.

23. The alcoxysilyl group-containing organic EL dye according to claim 22, wherein R₂ and R₃ each represent a thienyl group which may have a substituent, and wherein the substituent is an aromatic hydrocarbon group, an aliphatic hydrocarbon group, or a heterocyclic group, each of which may have a substituent.

24. A method for producing an alkoxysilyl group-containing organic EL dye according to claim 14, the method comprising a step of mixing the organic EL dye with a silane coupling agent, wherein the organic EL dye includes a reactive group selected from the group consisting of a succinimidyl ester group, an alcoholate group, an amino group, a mercapto group, hydroxyl-terminated polyoxyethylene group.

25. A fluorescent silica particles comprising condensates of an alkoxysilyl-containing organic EL dye according to claim 14.

26. The alcoxysilyl group-containing organic EL dye according to claim 14, wherein the imidazole derivative is represented by the following general formulae (10)-(14): 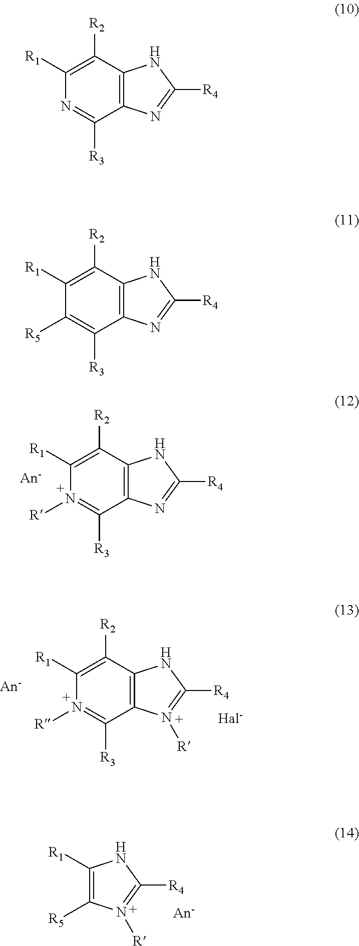
wherein
one of R₁ and R₄ in the formulae (10), (12) and (13) and one of R₁, R₄ and R₅ in the formulae (11) and (14) are each represented by the general formula L₃-M₃,
M₃ represents a nitrogen cation-containing group which is a pyridinium group, a secondary aminium group, a tertiary aminium group, a quaternary ammonium group, a piperidinium group, a piperazinium group, an imidazolium group, a thiazolium group, an oxazolium group, a quinolium group, a benzoimidazolium group, a benzothiazolium group, or a benzooxazolium group, each of which may have a substituent, or a nitrogen-containing group which is a pyridyl group, a secondary amino group, a tertiary amino group, a piperidyl group, a piperadyl group, an imidazolyl group, a thiazolyl group, an oxazolyl group, a quinolyl group, a benzoimidazolyl group, a benzothiazolyl group, or a benzooxazolyl group, each of which may have a substituent, L₃ represents a linker which is represented by —(CH═CR₆)ᵤ— and which connects M₃ with a center pyridine ring or a center benzene ring,
u represents an integer of from 1 to 5,
R₆ represents any one of a hydrogen atom; a linear or branched alkyl group which may have a substituent and has 1 to 6 carbon atoms; a sulfo group which may have a substituent; a heterocyclic group selected from the group consisting of an imidazolium group, a pyridinium group, and a furan group, each of which may have a substituent; an amino group selected from the group consisting of a secondary amino group, a tertiary amino group, and a quaternary amino group, each of which may have a substituent; a hydroxy group which may have a substituent; an alkoxy group which may have a substituent; an aldehyde group which may have a substituent; a carboxyl group which may have a substituent; and an aromatic group which may have a substituent,
the residues of R₁ and R₄ in the formula (10), (12) and (13), the residues of R₁, R₄ and R₅ in the formulae (11) and (14), and R₂ and R₃ each independently represent a hydrogen atom, a halogen atom, or an aromatic hydrocarbon group, an aliphatic hydrocarbon group, or a heterocyclic group, each of which may have a substituent,
X represents a nitrogen atom, a sulfur atom, an oxygen atom, a selenium atom, or a boron atom, each of which may have a substituent,
R′ represents an aliphatic hydrocarbon group including an alkyl group which may have an aromatic ring or an aromatic hydrocarbon group, and
An⁻ represents a halide ion, CF₃SO₃⁻, BF₄⁻, or PF₆⁻.
